(19) United States
(12) Patent Application Publication
(10) Pub. No.: US 2014/0349279 A1
(43) Pub. Date: Nov. 27, 2014

(54) 3D MICROFLUIDIC SYSTEM HAVING NESTED AREAS AND A BUILT-IN RESERVOIR, METHOD FOR THE PREPARING SAME, AND USES THEREOF

(71) Applicant: COMMISSARIAT À L'ÉNERGIE ATOMIQUE ET AUX ÉNERGIES ALTERNATIVES, PARIS (FR)

(72) Inventors: Thomas Berthelot, Les Ulis (FR); Hervé Volland, Orsay (FR)

(21) Appl. No.: 14/365,377

(22) PCT Filed: Dec. 14, 2012
(86) PCT No.: PCT/EP12/75644

§ 371 Date: Jun. 13, 2014

(30) Foreign Application Priority Data
Dec. 15, 2011 (FR) 11 61722
Oct. 23, 2012 (FR) 12 60083

(51) Int. Cl. G01N 33/52; G01N 33/573; B23P 19/00

(52) U.S. Cl. CPC G01N 33/525; B23P 19/00; G01N 33/573; G01N 2333/952
USPC 435/5; 29/428; 422/507; 436/501; 435/7.1; 435/7.4; 435/7.2; 435/7.92

(57) ABSTRACT

The present invention relates to a three dimensional (or 3D) microfluidic system comprising a plurality of layers stacked upon each other, characterised in that at least one of said layers consists of a 1^{st} and at least one 2^{nd} parts, distinct from each other, with the 2^{nd} part being porous and wettable by a solution of interest, nesting into a recess of the 1^{st} part being non-porous and/or non-wettable by said solution of interest, wherein said system can possibly have a built-in reservoir; a method for manufacturing the same and different uses thereof.

### TECHNICAL FIELD

The present invention relates to the field of diagnostic devices and in particular the field of such devices based on microfluidic systems.

Indeed, the present invention relates to a three dimensional (or 3D) microfluidic system comprising hydrophilic zones, hydrophobic zones nested into each other and possibly a built-in reservoir as well as a method for preparing such a system.

### STATE OF PRIOR ART

Immunological sensors as dipsticks are commonly used to detect numerous biological parameters but also to enable pathogens to be detected (“1D lateral-flow systems”). They are based on the lateral movement of fluids to be analysed through paper strips or dipsticks. These systems, although largely used, have limits as to their bioanalytic and fluidic capacities. In this format where the sample migrates along a single dimension, it is difficult to multiply the number of biological parameters detected. Indeed, the multiplication of detection lines or zones decreases the spatial resolution and can result in a wrong analysis of the result.

From 2008, new detection devices have arisen and have been designated “3D microfluidic devices”. The general concept of such new devices consists in creating hydrophilic microfluidic channels in a hydrophobic matrix and stacking the successive layers using a double sided Scotch® tape. Indeed, in 2008, the group of Professor G. Withesides (Harvard University) developed, based on the principle of immunological sensors as dipsticks, 3D microfluidic systems based on paper (and more generally based on a porous fibrous matrix) and SU-8 (photolithographic resin) [1-2]. Afterwards, similar systems but having a different embodiment have been developed by the group of W. Shen [3] or by the group of B. Lin [4].

These systems are in particular usable for colorimetric quantitative analyses with integrated calibration. Thus, the International Application WO 2011/000047 [5] contemplates the use of a 3D microfluidic system based on paper and comprising several hydrophilic test zones, wherein the sample to be tested and standard samples containing a known amount of the analyte to be assayed are deposited on different test zones and then the colorimetric intensity of the sample to be tested is compared with the standard colorimetric range obtained from standard samples. It is to be noted that in microfluidic systems such as described in [1] and [5], all the test zones evenly consist of the same material.

Up to now, these 3D microfluidic systems all rely on a sandwich structure made by stacking distinct layers from which at least one is a hydrophilic porous fibrous material locally hydrophobized following a given treatment. Thus, each layer consists of a same base material having locally zones with distinct physicochemical properties because of the physicochemical treatment(s) undergone.

More particularly, the technologies developed to make these layers coming as hydrophobic/hydrophilic matrices are derived from conventional lithographic techniques [1-2], “ink-jet” techniques by printing hydrophobic molecules [3] or wax printing techniques by a laser printer [4]. Their concepts therefore rely on the development in a porous layer of a hydrophilic network surrounded by a matrix that became hydrophobic, throughout its thickness, following the chemical modification by SU-8 wax or hydrophobic molecules impregnation. In particular, the International Application WO 2010/003188 provides a method for preparing a microfluidic system from a hydrophilic substrate [6]. This method comprises the steps of (i) hydrophobizing the surface of the substrate, (ii) depositing on the hydrophobized surface a mask having opening zones enabling the periphery of the microfluidic channels to be defined and (iii) applying, at the surface exposed via the opening zones, a treatment with irradiations so as to make the surface hydrophilic. During step (i), the surface is hydrophobized through absorption or adsorption of a solution of a hydrophobic substance such as defined in page 4, lines 29-33 in a volatile solvent. The treatment with irradiations which is in particular contemplated is a plasma treatment or a corona treatment.

Techniques used to obtain such structures are therefore dependent on the thickness of the porous material used. These embodiments are in particular non-applicable to thick porous fibrous matrices. Indeed, if the material to be treated to hydrophobize it is too thick, it will be impossible to create channels in the photolithographic resin impregnated matrix such as SU-8 through photolithography.

In the same way, it is impossible for the wax to impregnate the entire thickness of the thick support. Even if the wax is sprayed at high temperature, its cooling will be faster than its diffusion. Thus, even if the matrix is heated to enable the wax to be diffused, this will result in a strong design alteration or even in the plugging of the hydrophilic channel in its thickness depth thus preventing the 3D microfluidic system from being formed.

Consequently, for thick matrices, it is impossible to use the photolithography/photolithographic resin approach, the ink-jet printing of wax or hydrophobic molecules to obtain a fully hydrophobic matrix in contact with the hydrophilic zones, without leading to a system leakage sideways of the hydrophilic tracks and/or without resolution loss.

Furthermore, the use of photolithographic resin such as SU-8 and photolithographic methods are extremely money and time consuming, since these are multistep methods implying masking, developing, rinsing steps, etc. By way of example, the cost of 500 mL of SU-8 was 5000 [symbol] in 2011.

It is to be further noted that the hydrophilic matrix tracks such as prepared by the aforesaid methods are contacted with other chemicals such as resin, solvents, wax vapour, rinsing solvent, before they are used in the microfluidic system. This contact can lead to a possible contamination of the fluidic channel by by-products and lead to artefacts upon using the microfluidic system.

Finally, with the aforesaid methods, it is impossible to integrate in a same stage (between two double sided scotch tape) two or more porous hydrophilic material having a different nature and/or thickness like, for example, to introduce in the same stage a paper (cellulosic) fluidics and a glass fibre fluidics.

Therefore, the inventors have set themselves the aim to develop a method for manufacturing a 3D microfluidic device (i) independent of the thickness of the porous matrix, (ii) reducing the number of manufacturing steps with respect to methods of prior art thus providing a saving of time, (iii) at low cost, (iv) having a good lateral resolution and (v) that can integrate at least two fluidic systems based on porous materials having a different chemical nature.

Finally, in 1D and 3D microfluidic devices known up to now, depositing samples is made either by dipping part of the device into the sample, or by depositing the sample onto the device. In the latter case, the device is inserted in a cassette which enables a more or less high sample volume to be deposited.

Also, in order to simultaneously detect different biological parameters using dipsticks, plastic cassettes integrating several of them have been developed. These cassettes use the sample distribution on the different dipsticks and require a sample fractionation into as many aliquots as there are dipsticks. Indeed, the previously described systems enable the sample to be fractionated through capillarity which can thus interact with the different detection zones. This results in the analysis of small volumes for each parameter and thus a decrease in the detection threshold or the requirement to use high sample volumes in order to overcome this fractionation.

Therefore, the inventors have set themselves the aim to develop a method for manufacturing a 3D microfluidic device having the advantages as previously listed and some implementations of which also have a configuration enabling several parameters to be analysed without fractionating the sample in particular beyond a factor 2.

### DISCLOSURE OF THE INVENTION

The present invention allows the aim set by the inventors to be achieved and all or part of the technical problems of the 3D microfluidic systems of the state of the art to be solved.

More particularly, the system of the present invention and the method for preparing the same are based on the marquetry principle. Indeed, the sandwich structure results from the stack of layers wherein the hydrophilic porous fibrous layer locally hydrophobized is replaced by a mixed layer consisting of a hydrophobic support cut off beforehand according to the desired pattern, in particular by manual way or by cutting off with a CO₂ laser printer, wherein the porous hydrophilic material of interest is nested, the latter acting as a fluidic channel enabling aqueous solutions and samples to be transferred by capillarity effect into the plane and/or into the thickness of the final system. It should be noted that the association of a hydrophilic support with a porous hydrophobic material acting as a fluidic channel is also worth considering within the scope of the present invention.

The advantages of a 3D microfluidic system by “marquetry” according to the invention with respect to the systems developed in the state of the art are numerous:
decrease in the costs;
decrease in the time for developing the systems, because of the absence of contacting, diffusion, annealing, insolation, developing, washing, printing and/or heating steps;
no restriction as to the thickness of the material acting as a fluidic channel unlike previous systems that cannot use too thick matrices as previously explained;
no obligation about any treatment of the tracks of the matrix acting as fluidic channels for making them wettable by a solution of interest, the latter can be used in their “virgin” state, i.e. without ever having been contacted with other products such as resin, solvents, wax vapour or rinsing solvent;
great freedom as to the materials usable for the support part and for the fluidic channel type part(s).

Indeed, the fact that the support part and the fluidic channel type part(s) are nested and thus separable from each other enables numerous implementations which are impossible with the microfluidic systems of the state of the art to be contemplated. First, the support part and the fluidic channel type part(s) can be prepared from different materials. There is no restriction as to the materials usable for these different parts, unlike systems of the state of the art wherein the initial layer has to be in a single hydrophilic (or hydrophobic) material that can be hydrophobized (or hydrophilized) following a given treatment.

Further, when the microfluidic system according to the invention comprises several fluidic channel type parts on a same layer, all these parts or only some of them can be of an identical or different material. Thus, unlike systems of prior art, the present invention enables, in the same stage, 2 or more porous matrix types having a different chemical nature such as, for example, paper or glass fibre, to be introduced.

This latitude enables not only the physicochemistry of the fluidic channels to be adapted to the molecules to be analysed, but also the preparation of 3D microfluidic systems according to the invention to be considered in a “customized” way. By way of example, it is possible to nest in a support prepared beforehand, one (or more) fluidic channel type part(s) adapted to one (or more) molecule(s) of interest, depending on the desire of the user and at the time when the latter expresses it.

Thus, the present invention relates to a three dimensional (or 3D) microfluidic system comprising a plurality of layers stacked upon each other, wherein at least one of said layers consists of a 1^{st} and at least one 2^{nd} parts, distinct from each other, with the 2^{nd} part being porous and wettable by a solution of interest, nesting into a recess of the 1^{st} part being non-porous and/or non-wettable by said solution of interest, said 2^{nd} part acting as a fluidic channel providing transfer of said solution of interest by a capillarity effect into the plane and/or thickness of said system.

In the microfluidic system according to the invention, the 1^{st} part and the 2^{nd} part(s) are chemically different in particular because of their wettability and/or porosity difference.

In the microfluidic system according to the invention, the 2^{nd} part being porous and wettable by a solution of interest is previously cut off and comes as a shape adapted to be nested into the recess of the 1^{st} part.

Furthermore, the 1^{st} part makes up the support of the layer such as previously described and the 2^{nd} part(s) form(s) the fluidic channel via the stack of layers comprised in the microfluidic system according to the invention. Thus, the term “1^{st} part” and “support part” are equivalent and interchangeably usable. Further, the terms “2^{nd} part” and “fluidic channel type part” are equivalent and interchangeably usable.

As previously explained, at least one layer of the 3D microfluidic system according to the invention can comprise a single 2^{nd} part being porous and wettable by a solution of interest or at least two 2^{nd} parts, at least three 2^{nd} parts, at least four 2^{nd} parts, being porous and wettable by a solution of interest, having an identical or different nature and an identical or different shape, the assembly of these 2^{nd} parts nesting into as many recesses as 2^{nd} parts, being present at the support part of the layer.

In a particular embodiment, it is possible to keep at least one of the recesses of the 1^{st} part unfilled. Thus, the microfluidic system of this particular embodiment comprises a plurality of layers stacked upon each other, from which at least one layer consists of a 1^{st} part and at least one 2^{nd} part, distinct from each other, with the 2^{nd} part being porous and wettable by a solution of interest nesting into a recess of the 1^{st} part being non-porous and/or non-wettable by said solution of interest, said 1^{st} part further having at least one unfilled recess and said 2^{nd} part acting as a fluidic channel providing transfer of said solution of interest by a capillarity effect into the plane and/or thickness of said system.

Advantageously, in this particular embodiment, the microfluidic system can comprise at least one layer consisting of a 1^{st} part having at least one unfilled recess and at least one 2^{nd} part such as previously defined. In particular, the microfluidic system can comprise (i) at least one layer consisting of a 1^{st} part having an unfilled recess and a 2^{nd} part such as previously defined, (ii) at least one layer consisting of a 1^{st} part having an unfilled recess and two 2^{nd} parts such as previously defined and/or (iii) at least one layer consisting of a 1^{st} part having an unfilled recess and three 2^{nd} parts such as previously defined. Thus, the 3D microfluidic system according to the invention can comprise at least one layer (i) and at least one layer (ii), at least one layer (i) and at least one layer (iii) or at least one layer (ii) and at least one layer (iii). Moreover, the 3D microfluidic system according to the invention can comprise at least one layer (i), at least one layer (ii) and at least one layer (iii).

When the microfluidic system according to the invention has several layers each comprising at least an unfilled recess and in particular a single unfilled recess, the stack of these layers makes up a cannula and a reservoir which are built-in in the system. To do this, the recesses of these different layers are in fluid continuity towards each other. Advantageously, all the recesses or part of the same are centred on a common axis. Further advantageously, all the recesses or part of the same are centred on a common axis and have a cross-section with respect to the plane of the layer of an identical shape, their thickness being dependent on the thickness of the 1^{st} part wherein they have been prepared.

Within the scope of the present invention, by reservoir, it is meant a cavity defined by the recess of the 1^{st} part of a 1^{st} layer and bounded by at least the following layer. The cavity can be bounded by the single next layer: in this case, the reservoir is located at the layer of the upper end of the microfluidic system. Alternatively, the cavity is bounded both by the next layer and the previous layer: in this case, the reservoir is so-called built-in in the system. The next layer and/or the previous layer have at least one recess possibly filled at the recess forming the reservoir, both recesses being fluidly contacting with each other. In the case of a non built-in reservoir, only the next layer comprises such a recess wherein a 2^{nd} part such as previously defined is nested. In the case of a built-in reservoir, the previous layer comprises at least one recess which can be filled, partially filled or unfilled by a 2^{nd} part such as previously defined. In this embodiment, the following layer can also comprise at least one recess which can be filled, partially filled or unfilled by a 2^{nd} part such as previously defined, the recesses of the next and previous layers or parts of the same can be centred on a common axis or quite the opposite, non-centred on a common axis. Alternatively to this embodiment, the next layer may not comprise a recess fluidly contacting the reservoir and, in this case, the previous layer comprises at least two distinct recesses which can be, independently of each other, filled, partially filled or unfilled by a 2^{nd} part such as previously defined. In this particular alternative, the solution of interest “goes down” to the reservoir via a cannula or fluidic channel system and “goes up” to the upper layers via a distinct cannula or fluidic channel system.

Advantageously, the reservoir implemented in a microfluidic system according to the invention is fluidly contacting at least one fluidic channel such as previously defined and, to do this, in the recess of the 1^{st} part corresponding to said reservoir, an element can be located, of a hydrophilic and porous material (case of a hydrophilic solution of interest) or of a hydrophobic and porous material (case of a hydrophobic solution of interest). This element does not make up a 2^{nd} part as previously defined because it does not enable the recess made in the 1^{st} part to be fully filled.

The 1^{st} and 2^{nd} part(s) of at least one layer of the microfluidic system according to the invention are in particular defined relative to their wettability or non-wettability towards a solution of interest.

The wettability is defined by the contact angle (or connecting angle) that forms a drop of the solution of interest with the part at the deposit site of this drop.

Thus, when it is specified that the 2^{nd} part(s) is (are) wettable towards the solution of interest, this means generally that a drop of this deposited solution will form, relative to the deposit site, a contact angle having generally a value lower than 70° and in particular lower than 60° whereas, for the possibly non-wettable 1^{st} part, this means that the contact angle formed between a drop of the solution and this part has generally a value higher than 90° and in particular higher than 95°.

From a practical point of view, this means that, when the solution of interest is deposited onto the 2^{nd} part(s), it remains at this level without going into the non-wettable 1^{st} part.

From a chemical point of view, a liquid will wet the substrate that represents the part of the microfluidic system if it has a chemical affinity towards the same. Thus, a hydrophilic (or hydrophobic) substrate will be wettable toward hydrophilic (or hydrophobic) liquids.

It should be specified that the definition of wettability such as given above relates to the conventional definition of wettability relative to a liquid on a planar surface. Within the scope of the present invention, the chemical nature and in particular the wettability of the surface of the 2^{nd} part(s) intervenes in the transfer of the solution of interest which is made through capillarity but not only because the solution also passes through the open pores of the material of this (these) 2^{nd} part(s). Thus, the 2^{nd} part(s) can also be defined as permeable to the solution of interest. Finally, the 2^{nd} part(s) make(s) up a porous system through which the solution of interest flows, this flow being all the faster that the hydrophilicity/hydrophobicity of the porous material is compatible with the hydrophilicity/hydrophobicity of the solution of interest.

Within the scope of the present invention, by “solution of interest”, it is meant any natural or synthetic, hydrophilic or hydrophobic solution, which is desired to be analysed and/or purified on a 3D microfluidic system according to the present invention. It should be emphasized that the solution of interest implemented within the scope of the present invention should be neither a solvent of the 1^{st} part, nor a solvent of any of the 2^{nd} parts of the 3D microfluidic system.

Thus, this solution of interest can be a biological fluid; a plant fluid such as sap, nectar and root exudate; a sample in a culture medium or in a biological culture reactor such as a cell culture of higher eukaryotes, yeasts, fungi or algae; a liquid obtained from one or more animal or plant cell(s); a liquid obtained from an animal or plant tissue; a sample in a food matrix; a sample in a chemical reactor; municipal, river, pond, lake, sea or air-cooled tower water; a sample from a liquid industrial effluent; waste water coming in particular from intensive livestock or industries of the chemical, pharmaceutical, cosmetical or nuclear field; a pharmaceutical; a cosmetic; a fragrance; a soil sample or a mixture thereof.

The biological fluid is advantageously selected from the group consisting of blood such as whole blood or anticoagulated whole blood, blood serum, blood plasma, lymph, saliva, sputum, tears, sweat, semen, urine, feces, milk, cerebrospinal fluid, interstitial liquid, an isolated bone narrow fluid, a mucus or fluid from the respiratory, intestinal or genito-urinary tract, cell extracts, tissue extracts and organ extracts. Thus, the biological fluid can be any fluid naturally secreted or excreted from a human or animal body or any fluid recovered from a human or animal body, by any technique known to those skilled in the art such as extraction, sampling or washing. The recovery and isolation steps of these different fluids from the human or animal body are made prior to implementing the method according to the invention.

Furthermore, if one of the considered samplings does not enable a 3D microfluidic system according to the present invention to be implemented, for example because of its particularly solid nature, its concentration or elements it contains such as solid residues, wastes, suspension or interfering molecules, this implementation and in particular the detection method such as defined hereinafter further comprises a prior step of preparing the solution of interest with possibly dissolving the sample by the techniques known to those skilled in the art such as filtration, precipitation, dilution, distillation, blending, concentration, lysis, etc.

Different implementations with respect to the 1^{st} part of at least one layer of the 3D microfluidic system according to the invention are worth considering. Indeed, the latter can be:
porous and non-wettable by the solution of interest;
non-porous and wettable by the solution of interest; or
non-porous and non-wettable by the solution of interest.

Thus, for a hydrophilic solution of interest, the 1^{st} part (i.e. the support part) of at least one layer, in particular of one (or more) layer(s) and advantageously of all the layers of the microfluidic system according to the invention can be:
porous and hydrophobic;
non-porous and hydrophilic; or
non-porous and hydrophobic;

the 2^{nd} part(s) being, in the case of a solution of interest hydrophilic, hydrophilic and porous.

Advantageously, for a hydrophilic solution of interest, the support part of at least one layer, in particular of one (or more) layer(s) and advantageously of all the layers of the microfluidic system according to the invention is selected from the group consisting of a porous hydrophobic polymeric film; a membrane, a gel or a hydrophobic porous resin; a hydrophilic or hydrophobic non-porous polymeric film and a hydrophilic or hydrophobic non-porous membrane or resin.

In particular, by way of illustrating and non-limiting examples, for a hydrophilic solution of interest, the support part of at least one layer, in particular of one (or more) layer(s) and advantageously of all the layers of the microfluidic system according to the invention is selected from the group consisting of a porous or non-porous film of polyethylene terephthalate (PET); a porous or non-porous membrane of polyethylene (PE); a porous or non-porous membrane of polypropylene (PP); a porous or non-porous film, or porous or a non-porous membrane, containing fluorine such as a porous or non-porous film, or a porous or non-porous membrane of hydrophobic polyvinylidene fluoride (PVDF); a porous or non-porous membrane of polytetrafluoroethylene (PTFE); a porous or non-porous copolymeric film, comprising vinylidene fluoride and tetrafluoroethylene; a porous or non-porous copolymeric film comprising vinylidene fluoride and hexafluoropropylene; a porous or non-porous film of polymethyl methacrylate (PMMA); a porous or non-porous film of poly(n-butyl acetate); a porous or non-porous film of poly(benzyl methacrylate); a porous or non-porous film of poly(chlorotrifluoroethylene); a porous membrane and, in particular an ion exchange porous membrane, functionalized by hydrophobic groups; a styrene polymer such as an advantageously porous polystyrene resin; a porous or non-porous film of polyacrylonitrile; a porous or non-porous film, of polymethacrylonitrile; a porous or non-porous film, of polyimide such as Kapton® and a mixture thereof. By “hydrophobic group”, it is advantageously meant, within the scope of the present invention, a fluorinated group; an aryl group possibly substituted with one (or more) fluorine atom(s) and in particular a phenyl group possibly substituted with one (or more) fluorine atom(s); and an alkyl group possibly substituted with one (or more) fluorine atom(s).

Furthermore, for a hydrophilic solution of interest, the 2^{nd} hydrophilic and porous part(s) is (are) advantageously selected from the group consisting of a hydrophilic fibrous material, a tissue, a porous hydrophilic film, a hydrophilic gel or a porous membrane bearing or functionalized by hydrophilic groups. By “hydrophilic group”, it is meant, within the scope of the present invention, a group selected from a hydroxyl group, a carbonyl group, a trialkoxysilane group, an anionic group, a cationic group, a tertiary amine group, an epoxy group, an ester group, an amide group, an acid anhydride group, a phosphonic acid group, a sulfonic acid group, an ammonium group and possibly their conjugates bases.

More particularly, by way of illustrating and non-limitating examples, for a hydrophilic solution of interest, the hydrophilic and porous 2^{nd} part(s) is (are) advantageously selected from the group consisting of paper in particular of cellulosic nature; cotton paper; agarose; gelatin; cellulose; methylcellulose; carboxymethylcellulose; nitrocellulose; cellulose acetate ester; alginate; polyolefin; a porous membrane and, in particular an ion exchange porous membrane, functionalized advantageously by radiochemical grafting, by hydrophilic groups such as an NAFION membrane; a Sephadex type resin conditioned as a membrane or a PVDF membrane; a glass fibre fabric; a polyacrylamide gel; a sepharose gel or a mixture thereof.

Alternatively, for a hydrophobic solution of interest, the support part of at least one layer, in particular of one (or more) layer(s) and advantageously of all the layers of the microfluidic system according to the invention can be:
porous and hydrophilic;
non-porous and hydrophobic; or
non-porous and hydrophilic;

the 2^{nd} part(s) being, in the case of a solution of interest hydrophobic, hydrophobic and porous.

Advantageously, for a hydrophobic solution of interest, the support part of one (or more) layer(s) and advantageously of all the layers of the microfluidic system according to the invention is selected from the group consisting of a porous hydrophilic polymeric film; a membrane, a gel or a porous hydrophilic resin; a hydrophilic or hydrophobic non-porous polymeric film and a hydrophilic or hydrophobic non-porous membrane or resin.

In particular, by way of illustrating and non-limiting examples, for a hydrophobic solution of interest, the support part of at least one layer, in particular of one (or more) layer(s) and advantageously of all the layers of the microfluidic system according to the invention is selected from the group consisting of paper; cotton paper; agarose; gelatine; cellulose; methylcellulose; carboxymethylcellulose; nitrocellulose; cellulose acetate ester; alginate; polyolefin; a membrane and, in particular an ion exchange membrane, functionalized advantageously by radiochemical grafting, by hydrophilic groups such as an NAFION membrane; a Sephadex type resin conditioned as a membrane or a PVDF membrane; a glass fibre fabric; a non-porous film of polyethylene terephthalate (PET); a non-porous membrane of polyethylene (PE); a non-porous membrane of polypropylene (PP); a non-porous film of polyimide such as Kapton®; a polyacrylamide gel; a sepharose gel or a mixture thereof.

In particular, by way of illustrating and non-limiting examples, for a hydrophobic solution of interest, the 2^{nd} hydrophobic and porous part(s) is (are) selected from the group consisting of a hydrophobic fibrous material, a hydrophobic tissue, a porous hydrophobic film, a gel or a porous membrane bearing or functionalized by hydrophobic groups.

More particularly, by way of illustrating and non-limiting examples, for a hydrophobic solution of interest, the 2^{nd} hydrophobic and porous part(s) is (are) advantageously selected from the group consisting of paper hydrophobized by treatment, a porous film of polyethylene terephthalate (PET); a porous membrane of polyethylene (PE); a porous membrane of polypropylene (PP); a porous film or a porous membrane containing fluorine such as a porous film or a porous membrane of hydrophobic polyvinylidene fluoride (PVDF); a porous membrane of polytetrafluoroethylene (PTFE); a porous copolymeric film containing vinylidene fluoride and tetrafluoroethylene; a porous copolymeric film comprising vinylidene fluoride and hexafluoropropylene; a polyacrylamide gel; a porous film of polymethyl methacrylate (PMMA); a porous film of poly(n-butyl acetate); a porous film of poly(benzyl methacrylate); a porous film of poly(chlorotrifluoroethylene); a porous membrane and, in particular, an ion exchange porous membrane, functionalized by hydrophobic groups; a porous styrene polymer such as a porous polystyrene resin; a porous film of polyacrylonitrile; a porous film of polymethacrylonitrile and a mixture thereof.

Whether the 1^{st} part of a layer of the 3D microfluidic system according to the invention is hydrophobic or hydrophilic, it can have at least one self-adhesive face in direct contact with another layer of the system. Such a self-adhesive face can consist of a PET transparent element adhering on both sides, adhered on a 1^{st} part of a layer. This alternative enables two layers of the system to be secured together and/or tightness between these two layers to be ensured.

Whether the 2^{nd} part(s) of a layer of the 3D microfluidic system according to the invention is (are) hydrophobic or hydrophilic, it (they) can comprise at least one reagent or compound, able to detect and/or trap a component or analyte present in a solution of interest. Indeed, as explained hereinafter, the 3D microfluidic system according to the invention can be used in detection and/or purification methods. In other words, at least one reagent or compound is incorporated into the porous material of at least one 2^{nd} part implemented within the scope of the present invention.

When the material of this 2^{nd} part comprises at least one reagent or compound, the latter can be either distributed in the entire volume of the material or located in an accurate zone of the material. Advantageously, the reagents or compounds can be located at the surface of the pores or channels of the material. The reagents or compounds can be adsorbed at the surface of the pores or channels of this material and/or bonded to this surface by no-covalent bonds (hydrogen bonds or ionic bonds) and/or by covalent bonds.

When the binding between the reagent or compound and the material of the 2^{nd} part is low, the reagent or compound can be driven to lower layers by the flow of solution of interest, once the latter has been deposited onto the microfluidic system.

On the contrary, in the case of a strong binding, the reagent or compound remains in this material even in the presence of the solution of interest and the detection and/or trapping occur in this same layer. The same is true in the case of a reagent or compound non-covalently bonded but too bulky with respect to the pores and channels of the fluidic channel type part of the consecutive lower layer. To attach, by means of covalent bonds, the reagent or compound to the material of the 2^{nd} part, different methods, known to those skilled in the art, using spacer arms or not, are usable. Further, the experimental part hereinafter describes a method using cleavable aryl diazonium salts and based on the method described in the International Application WO 2008/078052 [7] and a method using aryl azide salts to attach reagents or compounds onto a material of the 2^{nd} part.

Further, the 3D microfluidic system according to the invention can comprise at least 2 layers, at least 3 layers, at least 4 layers, at least 5 layers, at least 10 layers, or even at least 50 layers, all or part of these layers can have a 1^{st} and at least one 2^{nd} parts such as previously defined.

In the present invention, the wording “upper layers” and “lower layers” concerns the support on which the microfluidic system according to the invention is possibly placed. Thus, the layer of the lower end corresponds to the layer directly contacting this support, the layer of the upper end corresponding to the layer most remote from this support.

These layers can have an identical or different thickness with respect to each other. Advantageously, the thickness of each layer is between 0.1 μm and 20 mm, in particular between 0.5 μm and 5 mm, typically between 1 μm and 1 mm, in particular between 2 μm and 400 μm and, more particularly, between 5 μm and 200 μm thus enabling a 3D microfluidic system having a thickness between 2 μm and 100 mm, typically between 5 μm and 50 mm, in particular between 10 μm and 25 mm and, in particular, between 40 μm and 10 mm to be obtained. It should be noted that, for a same layer of the 3D microfluidic system according to the invention, the thickness of the 2^{nd} part(s) has to be adapted as a function of the thickness of the 1^{st} part. Advantageously, for a same layer, the 1^{st} part and the 2^{nd} part(s) have substantially identical thicknesses.

Advantageously, all the layers included in the 3D microfluidic system according to the invention have a 1^{st} part and at least one 2^{nd} part such as previously defined. In a certain implementation, among the layers included in the 3D microfluidic system according to the invention having a 1^{st} part and at least a 2^{nd} part such as previously defined, one or more of these layers can comprise at least one recess, in the 1^{st} part, which is unfilled.

It is also possible that some of the layers included in the 3D microfluidic system according to the invention have a support part with one (or more) recess(es) without a fluidic channel type 2^{nd} part such as previously defined nested in the same. In other words, at least one layer of the stack of layers making up the microfluidic system according to the invention only consists of a support being non-porous and/or non-wettable by a solution of interest and recessed at one (or more) defined zone(s). It is clear that this (these) recess(es) make(s) part of the fluidic channel(s) the microfluidic system comprises.

When such a layer is included between two layers comprising a 1^{st} part and at least one 2^{nd} part such as previously defined, this layer could then be considered as a switch or a fluidic selection or distribution system. Indeed, as long as the 2^{nd} parts of the layers, separated by the layer without a 2^{nd} part, are not in contact, there is a discontinuity in the fluidic system and the liquid cannot pass therethrough, as explained in paragraph 84 of [1]. On the other hand, if a mechanical pressure action or a crushing onto this zone is performed as described in paragraph 83 of [1], both stages are then put into contact and the fluidic system is then connected and the solution of interest can pass therethrough.

A layer having a support part with one (or more) recess(es) without a fluidic channel type 2^{nd} part such as previously defined nested in the latter, is advantageously present, in the stack of layers which makes up the 3D microfluidic system according to the present invention, from the upper and lower layers of this stack. More particularly, such a layer is the 1^{st} layer of this stack (i.e. the layer on which the solution of interest is deposited) and/or the last layer of this stack. In this implementation, the recess(es) that the 1^{st} layer has (or layer of the upper end) serve(s) as a reservoir of solution of interest and the last layer (or layer of the lower end) can serve to keep the layer just above the same and in particular above the 2^{nd} part(s) the same comprises. In this implementation, the reservoir is located at the upper part of the device (i.e. at the 1^{st} layer), whereas, in the previously considered alternative implementing one or more layers having a 1^{st} part, at least one 2^{nd} part such as previously defined and at least one recess being unfilled at said 1^{st} part, the reservoir is advantageously at the lower layers, i.e. is built-in into the structure of the system.

The use of layers having particular properties as regards the number of 2^{nd} parts or as regards thickness, porosity and/or functionalization of the material making up these 2^{nd} parts enables these layers to be provided with a particular function. Some of these layers can have a function in the distribution and division of the solution of interest, in analysis, detection and/or purification.

Thus, the 3D microfluidic system according to the invention can in particular have at least one layer at least one 2^{nd} part of which such as previously defined and in particular the 2^{nd} part of which such as previously defined plays the role of biological dustbin. The latter enables all or part of the solution of interest to be recovered once the detection, quantification and/or purification of a given analyte are carried out.

Also generally and as described hereinafter, the shape of the recesses made in the 1^{st} part of the layer of the system according to the invention and thus that of the 2^{nd} part(s) possibly filling them vary a lot. In a particular implementation, the 3D microfluidic system according to the invention can in particular have at least one layer at least one 2^{nd} part of which as previously defined and in particular the 2^{nd} part of which as previously defined has a spiral or double spiral shape.

Further, it can be advantageous to control the thickness of at least one layer and/or the porosity of at least one 2^{nd} part of at least one layer of the 3D microfluidic system according to the invention. Those skilled in the art know different techniques enabling the porosity of hydrophilic or hydrophobic porous material such as previously described to be controlled. This control is in particular implemented during the preparation of the hydrophilic and porous material (or the hydrophobic and porous material). It can depend on conditions in particular the weaving conditions within the scope of a woven material and/or on reagents used during this preparation and in particular the addition of pore-forming agents in the reaction medium.

As regards the interest of such a control, there can be mentioned the example of the filtration of a solution of interest having the form of a complex medium comprising molecules, such as proteins, peptides, sugars modified or not, organic molecules taking part into the different signalling or metabolic ways, etc., in solution, cells but also cell debris such as membrane debris. The 3D microfluidic system according to the invention can, for example, be used for detecting the presence of a particular protein in this complex mixture. Thus, in order to avoid measurement artefacts, the complex medium can be deposited on the 2^{nd} part of the first layer which has a certain thickness and a controlled porosity (lower than the size of the cells and cell debris) in order to purify the sample and obtain at the outlet of this 2^{nd} part, only the medium containing the molecules, such as proteins, peptides, sugars modified or not, organic molecules taking part into the different signalling or metabolic ways, etc., the cells and cell debris being trapped into the material making up this part.

Another example is the deposition of a solution of interest containing previously lysed cells. The deposition of the solution onto the 2^{nd} part of the first layer which has a certain thickness and a controlled porosity enables the sample to be purified by keeping the membrane debris and organites trapped into the paper and will only let pass the contents internal to the cells such as proteins, DNAs or different metabolites.

Moreover, it is possible to have for a same 3D microfluidic system, 2^{nd} parts of different materials, being porous and wettable by a given solution of interest, materials, enabling molecules or organic materials having a different nature to be attached onto a same system. By way of examples, there can be zones or 2^{nd} parts containing materials being porous and wettable by a given solution of interest, being different by virtue of their chemical nature, by virtue of their thickness or by virtue of their porosity. Indeed, in a same 3D microfluidic system, different types of biological molecules such as proteins and cell elements can have to be analysed at the same time. However, these different elements to be analysed will not have the same physicochemical properties towards the porous matrix. Indeed, some could be adsorbed by electrostatic effect preventing any migration into the system and making the analysis impossible if it is this element that is desired to be analysed or quantified. It is in particular the case of some bacteria which are very strongly adsorbed into the nitrocellulose membranes thus preventing their migration within the system and making it inefficient for detection. On the contrary, if it is desired to get rid of this element for analysis, this phenomenon could then be used. Consequently, if it is desired to analyse a protein and a bacterium with a same 3D microfluidic system according to the invention and if the porous matrix used for the protein cannot serve to the bacterium (for the reasons set forth above), conduction channels having different natures should be used within the same stage, i.e. a same layer.

In a particular implementation of the microfluidic system according to the present invention, the latter, between at least two consecutive layers, has an element able to secure these two layers together and/or ensure the tightness between these two layers. Advantageously, such an identical or different element is present between all the layers of the microfluidic system according to the present invention.

This element can be in the form of a double sided Scotch®, a Saran microwave stretchable film type stretchable film or a derivative of a supported adhesion primary coat.

When double sided Scotch® tape or a stretchable film is implemented, the latter has at least one recess and in particular one (or more) recess(es) to ensure continuity of the fluidic channel between both layers it secures. Advantageously, one (or more) of these recesses is (are) filled with a material being porous and wettable by a solution of interest, which is identical to or different from the material of one the 2^{nd} part(s) of the layers it secures together. Alternatively, this (these) recess(es) is (are) left as such. However, as previously set forth for the layers of the system that can have a support part with at least one recess without any fluidic channel type 2^{nd} part such as previously defined nested, it can be required to mechanically act by pressure or crushing onto the unfilled recessed zones, to ensure the fluidic continuity of the layers separated by such an Scotch® tape or stretchable film.

The notion of “derivative of a supported adhesion primary coat” used within the scope of the present invention is directly related to the invention described in the International Application WO 2009/121944 [8]. Indeed, this application relates to a method for assembling two surfaces together. Within the scope of the present invention, both these surfaces are the lower surface of the layer and the upper surface of the consecutive layer. The derivative of a supported adhesion primary coat bridges both these surfaces by means of covalent bonds. Typically, such a supported adhesion primary coat is present on all or part of the 1^{st} part of at least one of both layers. It is advantageously a supported cleavable aryl salt and, in particular, a supported cleavable aryl diazonium salt of the following formula (I):

(surface)-(B)ₙ—R—N₂⁺,A⁻ (I)

wherein:
surface representing the surface of a 1^{st} part of a layer of a 3D microfluidic system according to the invention,
(B)ₙ represents a linker,
n is 0 or 1,
A represents a monovalent anion, and
R represents an aryl group.

B can represent a single entity, at least two identical or different entities or even a finish such as described in [8]. Further, all the alternatives and embodiments contemplated in [8] for A and R are usable within the scope of the present invention.

The present invention also relates to a device comprising a microfluidic system such as previously defined. Such a device can be in the form of a plate, a box or casing of plastic or ceramic wherein the 3D microfluidic system according to the invention is placed. The 3D microfluidic system or device according to the invention can be built-in into a suitable packaging with in particular instructions of use but also in a more complicated system where the item can be, for example, the 3D microfluidic system or device according to the invention.

The present invention also relates to a method for manufacturing a 3D microfluidic system such as previously defined, said method comprising the steps of:

a₁) preparing, in a layer of a material being non-porous and/or non-wettable by a solution of interest, a recess;

b₁) cutting off, in a layer of a material being porous and wettable by said solution of interest in particular such as previously defined, at least one shape in accordance with the recess prepared in step (a₁);

c₁) nesting into the recess prepared in step (a₁) the shape cut off in step (b₁), whereby a layer of said 3D microfluidic system is obtained;

d₁) possibly repeating steps (a₁), (b₁) and (c₁); and

e₁) assembling the different layers of the 3D microfluidic system.

The aim of step (a₁) of the method is to prepare the 1^{st} part of a layer of the 3D microfluidic system. This recess can be achieved by any cutting off technique known to those skilled in the art. Advantageously, the latter is selected from a cutting off by selective chemical attack, a physical cutting off of the reactive ion etching (RIE) type, a manual cutting off such as drawing and a laser cutting such as pulsed laser with in particular a YAG (Yttrium Aluminium Garnet) type source or a continuous laser in particular a CO₂ source laser. These lasers enable a resolution in the order of 5 to 10 μm to be obtained.

During step (a₁), at least one other recess can be prepared in the layer of the material being non-porous and/or non-wettable by a solution of interest. This recess can be subsequently filled upon implementing steps (b₁) and (c₁) or, on the contrary, remained as such, i.e. unfilled.

During step (a₁), the recess(es) prepared can have various shapes. By way of shapes worth considering, a recess whose cross-section with respect to the plan of the layer is circular, oval, square, rectangular, triangular, star-, cross-, stick-, spiral- or double spiral-shaped can be mentioned. On a same layer, two recesses can have an identical shape or different shapes. Further, two materials filling two recesses each belonging to two successive layers and in fluid continuity to each other can have a cross-section with respect to the plane of the layer having an identical shape or different shapes.

The use of a recess whose cross-section with respect to the plane of the layer is in spiral or double spiral shape can have a definite advantage at the layer at least one 2^{nd} part of which is implemented in detection and/or purification methods. Indeed, such a configuration enables several detection zones (i.e. zones comprising at least one reagent or compound, able to detect and/or trap a component or analyte present in a solution of interest) to be carried out. Thus, several components or analytes can be identified from a same sample of solution of interest and this without requiring a fractionation of said sample.

Step (b₁) of the method according to the present invention consists in preparing at least one 2^{nd} part of a layer of the 3D microfluidic system according to the present invention. By “shape in accordance with the recess”, it is meant not only a shape perfectly nesting into the recess prepared in step (a₁) but also a shape the thickness of which is substantially equal to the thickness of the layer implemented in step (a₁). Advantageously, the cutting off implemented in step (b₁) is also selected from a cutting off by selective chemical attack, a physical cutting off of the reactive ion etching type, a manual cutting off and a laser cutting off such as previously defined.

Steps (a₁, (b₁) and (c₁) are repeated as many times as the 3D microfluidic system comprises layers with a 1^{st} part and at least one 2^{nd} part as previously defined.

The layer obtained following the nesting step (c₁) can be defined as a 2D microfluidic system because it is built-in into the plane of the support system.

The nesting of step (c₁) and assembly of step (e₁) can be manually or automatically carried out.

During step (e₁), the layers to be assembled can comprise not only one (or more) layer(s) such as prepared by implementing steps (a₁), (b₁) and (c₁) but also one (or more) layer(s) prepared by the single implementation of step (a₁) (i.e. one (or more) layer(s) consisting of a material being non-porous and/or non-wettable for a liquid of interest and having one (or more) recess(es) left as such) and/or one (or more) layer(s) of an adhesive such a previously defined.

In the particular implementation wherein the 3D microfluidic system according to the present invention comprises a derivative of supported adhesion primary coat, the preparation method comprises the steps of:

a₁′) preparating, in a layer of a material being non-porous and/or non-wettable by a solution of interest in particular such as previously defined and the surface of which has a supported adhesion primary coat and in particular the supported cleavable aryl salt, a recess;

b₁) cutting off, in a layer of a material being porous and wettable by said solution of interest in particular such as previously defined, at least one shape in accordance with the recess prepared in step (a₁′);

c₁) nesting into the recess prepared in step (a₁′) the shape cut off in step (b₁), whereby a layer of said 3D microfluidic system is obtained;

d₁) possibly repeating steps (a₁′), (b₁) and (c₁); and

e₁′) assembling the different layers of the 3D microfluidic system by subjecting the surface of the material being non-porous and/or non-wettable by a solution of interest to non-electrochemical conditions to obtain, from the supported adhesion primary coat and in particular from the supported cleavable aryl salt, on said surface, at least one radical and/or ionic entity and by contacting said surface having at least one radical and/or ionic entity thus obtained with the surface of the consecutive layer.

During step (a₁′), the functionalization of the surface of the material by a supported adhesion primary coat can be made prior to or after the recess of the material.

Steps (b₁), (c₁) and (d₁) are as previously defined. Further, the previously contemplated implementations for step (a₁) apply mutatis mutandis to step (a₁′).

The non-electrochemical conditions usable in step (e₁′) are as defined in [8]. It should be however noted that it is desirable that these conditions do not implement solutions likely to act onto the materials making up the layers of the microfluidic system.

Thus, advantageously, the non-electrochemical conditions usable in step (e₁′) are photochemical non-electrochemical conditions and consist in submitting the supported adhesion primary coat and in particular the supported cleavable salt to an irradiation (or insolation) in the UV or visible range.

The wavelength employed in step (e₁′) of the method will be selected, without any inventive effort, as a function of the adhesion primary coat and in particular the cleavable aryl salt used. Any wavelength belonging to the UV range or visible range is usable within the scope of the present invention. Advantageously, the wavelength applied is between 300 and 700 nm, in particular between 350 and 600 nm and, in particular, between 380 and 550 nm.

The microfluidic system offers a wide diversity as to the configurations worth considering. For the simplest configurations, the solution of interest is deposited onto the 1^{st} layer of the system and is recovered at a lower layer and in particular at the last layer. When such a device is used in detection or purification, the detection can also be implemented at the lower layer and in particular at the last layer.

Alternatively and in particular when the microfluidic system has a built-in reservoir therein, the solution of interest can be in contact with or pass through several layers several times, a 1^{st} time to go to the reservoir in a layer internal to the system (descending direction), a 2^{nd} time to go from the reservoir to the analysis or detection layer, the latter being advantageously the 1^{st} layer of the system (ascending direction) and a 3^{rd} and last time to go from the analysis layer to a lower layer and in particular the last layer which plays a “dustbin” role but also has a role in capillarity.

Whatever the configuration contemplated, it is important to note that the conveyance of the solution of interest in or through the layers of the system is only made by gravity and/or capillarity, no pump type element in particular of the mechanical type is required for this conveyance.

The present invention also relates to the use of a 3D microfluidic system such as previously defined or likely to be prepared by a manufacturing method such as previously defined for detecting and possibly quantifying at least one analyte possibly present in the solution of interest or a gas fluid of interest. By “gas fluid of interest”, it is advantageously meant an air sample or a sample from a gas industrial effluent.

In other words, the present invention relates to a method for detecting and possibly quantifying at least one analyte possibly present in a solution of interest or a gas fluid of interest, comprising the steps of:

a₂) possibly preparing a 3D microfluidic system able to detect said analyte according to a manufacturing method such as previously defined;

b₂) depositing said solution of interest onto the 3D microfluidic system possibly prepared in step (a₂) or contacting said gas fluid of interest with said 3D microfluidic system possibly prepared in step (a₂); and

c₂) detecting and possibly quantifying said analyte possibly present.

The analyte to be detected and possibly to be quantified is present in the solution of interest or the gas fluid such as previously defined and can be selected from the group consisting of a biological molecule of interest; a pharmacological molecule of interest; a toxin; a carbohydrate; a peptide; a protein; a glycoprotein; an enzyme; an enzymatic substrate; a nuclear or membrane receptor; an agonist or antagonist of a nuclear or membrane receptor; a hormone; a polyclonal or monoclonal antibody; an antibody fragment such as a Fab, F(ab′)₂, Fv fragment or a hypervariable domain or CDR (Complementarity Determining Region); a nucleotide molecule; an advantageously organic pollutant of water; an advantageously organic pollutant of air; a bacterium and a virus.

The expression “nucleotide molecule” used in the present invention is equivalent to the following terms and expressions: “nucleic acid”, “polynucleotide”, “nucleotide sequence”, “polynucleotide sequence”. By “nucleotide molecule”, it is meant, within the scope of the present invention, a chromosome; a gene; a regulating polynucleotide; a single-stranded or double-stranded, genomic, chromosomal, chroroplastic, plasmid, mitochondrial, recombinant or complementary DNA; a total RNA; a messenger RNA; a ribosomal RNA (or ribozyme); a transfer RNA; a sequence acting as an aptamer; a portion or fragment thereof.

The reagent used to functionalize the microfluidic system implemented within the scope of the present invention is any molecule capable of forming with the analyte to be detected a binding pair, the reagent and the analyte corresponding to the two partners of this binding pair. The bonds implemented in the analyte-reagent binding are either non-covalent low energy bonds such as hydrogen bonds or Van der Waals bonds, or covalent bonds type high energy bonds.

The reagent used thus depends on the analyte to be detected. Depending on this analyte, those skilled in the art will be able, without any inventive effort, to select the most suitable reagent. It can be selected from the group consisting of a probe molecule; a carbohydrate; a peptide; a protein; a glycoprotein; an enzyme; an enzymatic substrate; a membrane or nuclear receptor; an agonist or antagonist of a membrane or nuclear receptor; a toxin; a polyclonal or monoclonal antibody; an antibody fragment such as a Fab, F(ab′)₂, Fv fragment or a hypervariable domain (or CDR (Complementarity Determining Region)); a nucleotide molecule such as previously defined; a peptide nucleic acid and an aptamer such as a DNA aptamer or an RNA aptamer.

By “probe molecule”, it is meant, within the scope of the present invention, a molecule specific to one (or more) analyte(s) for which a contact with at least one of these analytes results in at least one modification of the spectral properties of this probe molecule.

The probe molecule implemented within the scope of the present invention is a molecule which has fluorogenic or chromogenic properties, that is it becomes fluorescent or colours when it interacts with at least one specific analyte. Generally, the interaction of the probe molecule with at least one specific analyte produces a detectable optical signal. The interaction can consist in creating an irreversible and selective bond, in particular of the covalent bond type between the probe molecule and at least one specific analyte.

Those skilled in the art know different molecules usable within the scope of the present invention and known to detect specific analytes such as an aldehyde, formaldehyde, acetaldehyde, naphthalene, a primary amine in particular an aromatic one, indole, scatole, tryptophan, urobilinogen, pyrrole, benzene, toluene, xylene, styrene or naphthalene. Such probe molecules are in particular selected from enaminomes and the corresponding β-diketone/amine couples, imines and hydrazines, 4-aminopent-3-en-2-one, croconic acid and aldehyde function probe molecules as p-dimethylaminobenzaldehyde, p-dimethylaminocinnamaldehyde, p-methoxybenzaldehyde and 4-methoxy-1-naphthaldehyde, mixtures and salts derived from these compounds.

The use of a 3D microfluidic system according to the invention or likely to be prepared by a manufacturing method according to the present invention to detect and possibly quantify at least one analyte such as previously defined can thus be applied in the field of biochemistry, microbiology, in the field of medical diagnostic, in the nuclear field, in the quality control field in the food-processing industry, in the field of illegal substance screening, in the field of defence and/or biodefence; in the field of veterinary, environmental and/or sanitary control and/or in the field of perfume, cosmetics and/or aromas.

Step (a₂), when it has to be implemented, consists in preparing a 3D microfluidic system able to detect at least one analyte, i.e. a 3D microfluidic system comprising at least a reagent able to recognize specifically said analyte.

During step (b₂) of the method according to the present invention, the deposition of the solution of interest consists in depositing one (or more) drop(s) of said solution of interest at one (or more) fluidic channel(s) present at the surface of the 3D microfluidic system. The volume of solution of interest deposited onto each fluidic channel is between 1 μl and 10 ml, in particular between 20 μl and 5 ml, in particular between 400 μl and 2 ml.

Step (c₂) of the method according to the present invention consists in detecting and possibly quantifying the signal emitted by
the analyte,
the binding pair formed between said analyte and the reagent present in the 3D microfluidic system possibly prepared in step (a₂),
a secondary reagent able to recognize the analyte or the binding pair formed between said analyte and the reagent.

The secondary reagent can be selected from the group consisting of a carbohydrate; a peptide; a protein; a glycoprotein; an enzyme; an enzymatic substrate; a membrane or nuclear receptor; an agonist or antagonist of a membrane or nuclear receptor; a toxin; a polyclonal or monoclonal antibody; an antibody fragment such as a Fab, F(ab′)₂, Fv fragment or a hypervariable domain (or CDR (Complementarity Determining Region)); a nucleotide molecule such as previously defined; a peptide nucleic acid and an aptamer such as a DNA aptamer or a RNA aptamer.

In order to carry out the detection, the analyte and/or the secondary agent have at least one readily detectable group, that is a group which emits a measurable (enzymatic, fluorescent, chromogenic, radioactive . . . ) signal or which enables a measurable signal to be formed.

In a 1^{st} implementation of the readily detectable group, the latter can be an enzyme or a molecule capable of generating a detectable signal and possibly quantifiable under particular conditions as upon placing an adapted substrate. By way of illustrating and non-limiting examples, biotin, dioxygenin, 5-bromodeoxyuridine, an alkaline phosphatase, a peroxidase, a glucose amylase and a lysozyme can be mentioned.

In a 2^{nd} implementation of the readily detectable group, the latter can be a fluorescent, chemifluorescent or bioluminescent label such as cyanine and its derivatives, fluorescein and its derivatives, rhodamine and its derivatives, GFP (Green Fluorescent Protein) and its derivatives, coumarine and its derivatives and umbelliferon; luminol; luciferase and luciferin.

In a 3^{rd} implementation of the readily detectable group, the latter can be a radioactive label or isotope or an organic group containing at least one radioactive label or isotope. Such a radioactive label or isotope is in particular iodine-123, iodine-125, iodine-126, iodine-133, l'iode-131, iodine-124, indium-111, indium-113m, bromine-77, bromine-76, gallium-67, gallium-68, ruthenium-95, ruthenium-97, technetium-99m, fluorine-19, fluorine-18, carbon-13, nitrogen-15, oxygen-17, oxygen-15, oxygen-14, scandium-47, tellurium-122m, thulium-165, yttrium-199, copper-64, copper-62, carbon-11, nitrogen-13, gadolidium-68 and rubidium-82.

In a 4^{th} implementation of the readily detectable group, the latter can be a paramagnetic or ferromagnetic label such as 2,2,6,6-tetramethylpiperidine N-oxide (TEMPO) or a complex capable of chelating paramagnetic or ferromagnetic ions as Gd³⁺, Cu²⁺, Fe³⁺, Fe²⁺ and Mn²⁺.

In a 5^{th} implementation of the readily detectable group, the latter can be a gold particle or a dense compound such as a nanoparticle and, in particular, by way of example, a ferric oxide or gold (nano) particle.

Thus, the detection during step (c₂) according to the invention can be performed in particular by colorimetric, (chemi)fluorescence, bioluminescence, radioactive, ferromagnetic, paramagnetic and/or electric measurements.

Advantageously, this detection is performed by measuring the absorbance, (chemi)fluorescence, (bio)luminescence, radioactivity, ferromagnetism or paramagnetism variation of the 3D microfluidic system according to the present invention or at least one of the spots it has when this system is contacted with a solution of interest containing the analyte(s) such as previously defined. By way of examples, when the detection is performed by optical measurement, the wavelength for which the absorbance, (chemi)fluorescence or (bio)luminescence of the reagent such as a probe molecule, or the product formed by the interaction or reaction between the reagent and the detected analyte is the highest is preferably selected.

Alternatively, this detection is performed by measuring the variation in the electrical signal emitted by the 3D microfluidic system according to the present invention or at least, one of the spots it has when the system is contacted with a solution of interest containing the analyte(s) such as previously defined.

The electrical and possibly optical, colorimetric, (chemi)fluorescent, bioluminescent, radioactive, ferromagnetic or paramagnetic detection can require that the 3D microfluidic system according to the present invention is deposited onto a detection system including electric tracks in particular of copper or gold or that the detection stage of the 3D microfluidic system according to the present invention comprises electrical tracks in particular of copper or gold, protected from oxidation in particular by depositing parylene. These electric connections can be useful for electric detections or for integrating more complex systems such as diodes to stimulate fluorescence or phosphorescence of the reagent such as a probe molecule, or the product formed by the interaction or reaction between the reagent and the analyte detected on the detection site.

Regardless of the nature of the measurement performed, the latter can be compared to the measurement obtained from calibrated solutions of known analytes to directly give information about quantity and/or nature of the analyte(s) contained in the solution of interest.

In a particular implementation, the exposition of the 3D microfluidic system according to the present invention to a liquid solution can give rise to a visual, photographic observation or via a scanner. In this case, a comparison of the coloration intensity developed with those previously set during a calibration for a predefined concentration range, enables the analyte concentration to be coarsely deduced. For a fine quantitative measurement, a measurement of the absorption variation in a point or in a wide spectral range as a function of the contact time is necessary in order to determine a formation rate of the coloured complex. The value of this rate can be compared with those obtained for a control range under the same conditions.

Prior to or during the detection step (c₂), solutions other than the solution of interest or the gas fluid may have to be deposited onto the 3D microfluidic system according to the present invention. Indeed, washing solutions to remove any non-specific binding implying the reagent, a solution containing the secondary reagent or solutions containing a substrate adapted to an enzyme carried by the analyte or the secondary reagent may have to be deposited onto the 3D microfluidic system according to the invention.

The present invention finally relates to the use of a 3D microfluidic system such as previously defined or likely to be prepared by a manufacturing method such as previously defined to purify an analyte present in a solution of interest or a gas fluid of interest. In other words, the present invention relates to a method for purifying at least one analyte present in a solution of interest or a gas fluid of interest, comprising the steps of:

a₃) possibly preparing a 3D microfluidic system able to purify said analyte according to a manufacturing method such as previously defined;

b₃) depositing said solution of interest onto said 3D microfluidic system possibly prepared in step (a₃) or contacting said gas fluid of interest with said 3D microfluidic system possibly prepared in step (a₃); and

c₃) purifying said analyte.

Everything that has been described, within the scope of the detection and optional quantification method, as regards step (a₂), step (b₂), the analyte to be detected and possibly to be quantified and the solution of interest is also applicable mutatis mutandis to step (a₃), step (b₃), the analyte to be purified and the solution of interest in the purification method according to the present invention.

Indeed, in a 1^{st} implementation, the purification method according to the invention can require the use of a microfluidic system comprising at least one reagent able to recognize the analyte to be specifically purified. The purification during step (c₃) is performed by recovering the analyte of the binding pair formed by the latter and the reagent.

In a 2^{nd} implementation, the purification method according to the invention does not implement a specific reagent of the analyte such as previously defined. But, in this implementation, the purification is made by gradually removing components of the solution of interest, other than the analyte to be purified. Thus, step (a₃), when it has to be implemented, consists in preparing a 3D microfluidic system one (or more) layer(s) of which comprise(s) one (or more) compound(s) able to trap one (or more) of the components of the solution of interest, other than the analyte to be purified. In this implementation, step (c₃) of the method consists in recovering the analyte at least one lower layer of the 3D microfluidic system.

Within the scope of the use of the 3D microfluidic system according to the present invention in the field of purification, porous polymeric membranes having anion or cation exchange groups can be implemented to carry out a purification by ion exchange chromatography. It is also possible to use porous polymeric membranes or other porous materials modified beforehand by organic or biological specific molecules in order to perform affinity chromatography.

Further characteristics and advantages of the present invention will be further apparent to those skilled in the art upon reading examples given below by way of illustrating and non-limiting way, in reference to the appended figures.

### BRIEF DESCRIPTION OF THE DRAWINGS

FIG. 1 is a schematic view of different support parts, usable in a 3D microfluidic system according to the invention.

FIG. 2 is a schematic view of different fluidic channel type parts, usable in a 3D microfluidic system according to the invention.

FIG. 3 is a schematic view of a microfluidic system according to the invention with the different layers making it up, the drop symbolising the solution of interest to be analysed or purified and the arrow the migration direction of the sample.

FIG. 4 presents a casing wherein the 3D microfluidic system according to the invention can be inserted.

FIG. 5 presents results likely to be obtained for different solutions of interest (FIGS. 5B, 5C, 5D, 5E and 5F) using a microfluidic system according to the invention such as described in FIG. 5A.

FIG. 6 presents the different stages (FIGS. 6A, 6B and 6C) with the 2^{nd} part nested into the 1^{st} support before assembly of a 3 stage (or layer) 3D microfluidic system.

FIG. 7 presents the different stages (FIGS. 7A, 7B, 7C, 7D and 7E) with the 2^{nd} part(s) nested into the 1^{st} support before assembly of a 5 stage (or layer) 3D microfluidic system.

FIG. 8 presents a global system after assembly with FIG. 8A presenting the top of the system at which the solution of interest to be analysed is introduced and FIG. 8B the stage for reading the results.

FIG. 9 is a schematic view of a microfluidic system according to the invention used for detecting botulinum toxin A (FIG. 9A) and the layer for detecting this system (FIG. 9B).

FIG. 10 presents the results obtained with a solution of interest containing (FIG. 10B) or not (FIG. 10A) botulinum toxin A.

FIG. 11 is a representation, layer by layer, of the elements of porous hydrophobic material and elements of porous hydrophilic material, alone or associated together making up a 3D microfluidic system having a built-in reservoir and a double spiral.

FIG. 12 is the representation of the 3D microfluidic system having a built-in reservoir and a double spiral obtained from the elements presented in FIG. 11.

FIG. 13 is a diagram of the migration of the sample into the empty cannula and hydrophilic porous channels of the microfluidic system of FIG. 12.

FIG. 14 is a top view of the 3D microfluidic system having a built-in reservoir and a double spiral (layer (1)), once the sample of interest has been introduced in the same.

### DETAILED DISCLOSURE OF PARTICULAR EMBODIMENTS

I. Preparation of a 3D Microfluidic System According to the Invention.

The 3D microfluidic system according to the present invention described hereinafter is a system adapted for hydrophilic solutions of interest.

I.1. Preparation of Support Parts of the Layers of the System.

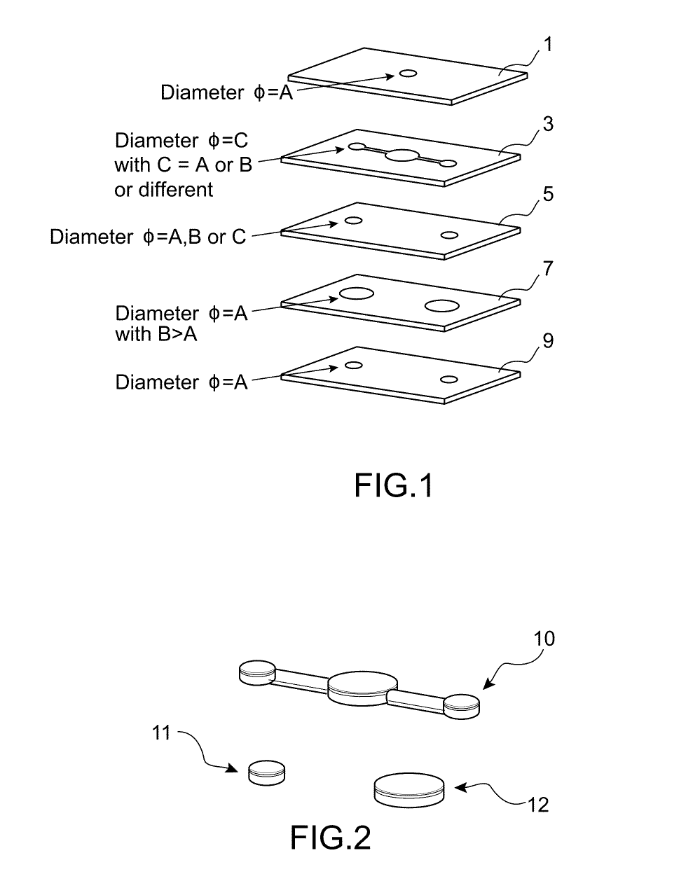

FIG. 1 presents the “negatives” (1, 3, 5, 7 and 9) performed in a PET type polymeric sheet and with one or two recess(es) per negative, having identical or different shapes, obtained after a drawing type manual cutting off or by a CO₂ laser printer.

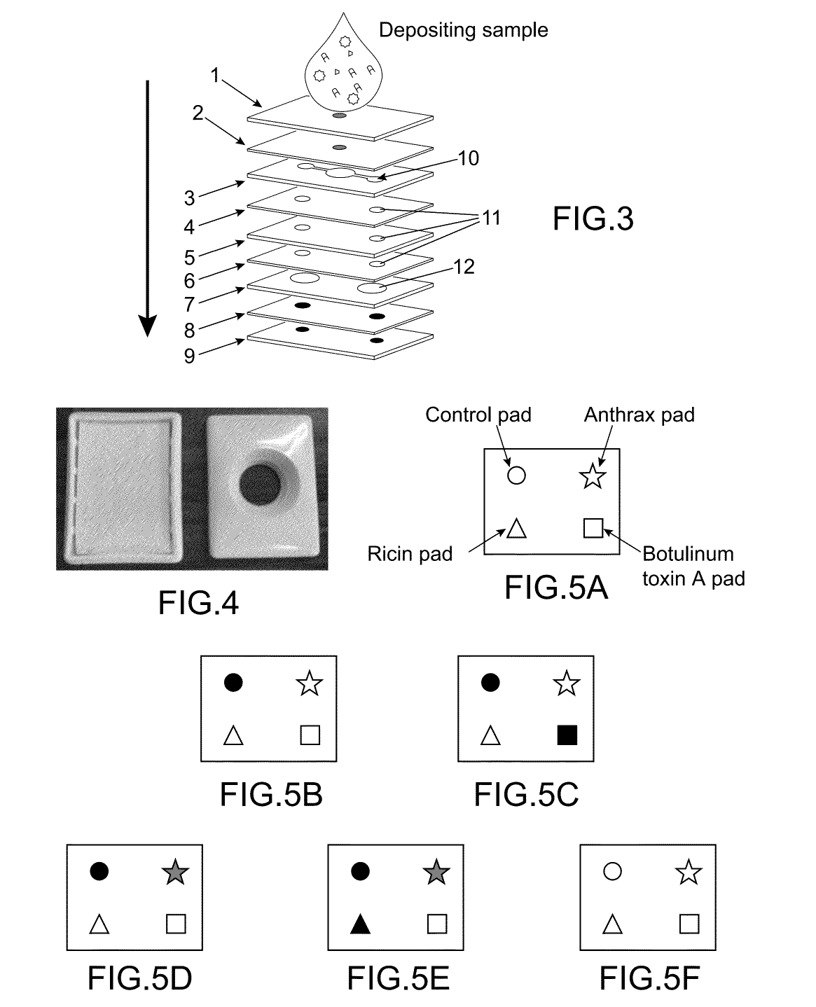

Three of these 5 “negatives” form the support part such as previously defined of the layers of the system (cf. FIG. 3).

I.2. Preparation of the “Fluidic Channel” Type Parts of the Layers of the System.

FIG. 2 presents the “positives” (10, 11 and 12) performed in a paper or glass fibre sheet, obtained after a drawing type manual cutting off or by a CO₂ laser printer.

These 3 types of “positives” form the fluidic channel type parts which are nested into the recesses of the “negatives” prepared at point 1.1.

I.3. Assembly of the System and System Thus Obtained.

The assembly of the device is made by:

1/ nesting the cut off hydrophilic part (“positive”) into the hydrophobic material (“negative”), the whole making up a mixed layer of the system according to the invention;

2/ adhering to the surface of this mixed layer of the double sided adhesive tape to leave recesses at the fluidic phase;

3/ positioning a new mixed layer onto the surface of the adhesive tape.

Steps 2 and 3 can be repeated as many times as required.

A 3D microfluidic system that can be obtained from the “negatives” and “positives” of FIGS. 1 and 2 is presented in FIG. 3.

The layers (1) and (9) of this system do not make up layers with a 1^{st} and at least one 2^{nd} parts such as defined in the present invention. Indeed, the recesses of the support part are left as such, without a “positive” nested therein. On the other hand, layers (3), (5) and (7) wherein a “positive” (10), two “positives” (11) and two “positives” (12) are respectively nested are actually layers with a 1^{st} and at least one 2^{nd} parts such as defined in the present invention.

Layers (2), (4), (6) and (8) represent the double sided Scotch® tape respectively placed between layers (1) and (3), (3) and (5), (5) and (7) and (7) and (9). These layers present recess zones to ensure a continuity of the fluidic channel. Some of the recesses are left as such, see layers (2) and (8), whereas, in the others, “positives” of hydrophilic material are nested. Thus, two “positives” (11) are nested into the Scotch® tape, see layers (4) and (6).

In the microfluidic system presented in FIG. 3, the layers of hydrophobic material have well determined functions with:
layer (1) which is the layer on which the sample of the solution of interest to be analysed or purified represented by a drop is deposited;
layer (3) which is a layer enabling the sample to be divided, in the present case, into two;
layers (5) and (7) corresponding to the analysis stages; and
layer (9) which contributes to support the hydrophilic parts of the upper stages and in particular of stage (7).

All these layers are deposited onto adsorbent paper to keep migration and the entire device is inserted in a small plastic box such as presented in FIG. 4.

II. Example of a Multiparameter 3D Microfluidic System According to the Invention.

FIG. 5A presents a microfluidic system according to the invention with 4 pads, having different shapes for facilitating in particular the reading of results:
with a control pad;
a pad the fluidic channel part of which has in the analysis layer anti-anthrax antibodies and thus able to develop, in the solution of interest to be analysed, the presence of anthrax “Anthrax Pad”;
a pad the fluidic channel part of which has in the analysis layer anti-ricin antibodies and thus able to develop the presence of ricin in the hydrophilic part “Ricin Pad”; and
a pad the fluidic channel type part of which has in the analysis layer anti-botulinum toxin A antibodies and thus able to develop the presence of botulinum toxin A “Botulinum Toxin A Pad”.

FIGS. 5B, 5C, 5D, 5E and 5F present the theoretical result, obtained for a negative sample for all the 3 agents tested; a positive sample for botulinum toxin A; a positive sample for anthrax; a positive sample for anthrax and for ricin; and a sample made under unsuitable conditions.

III. Exemplary Development of a System Having a Multi-Pad Reading Stage.

III.1. 4 Pad Reading Stage.

The different stages making up the 1^{st} support part are made of PET sheets 100 μm thick. The different stages have been obtained after cutting off with a CO₂ laser (Laser GCC Pro Spirit 25 watts). The design has been performed on Corel Draw 5 software.

The 2^{nd} part (fluidic system) has been obtained after cutting off CF1 Whatman paper by a CO₂ laser (Laser GCC Pro Spirit 25 watts). The design has been performed on Corel Draw 5 software.

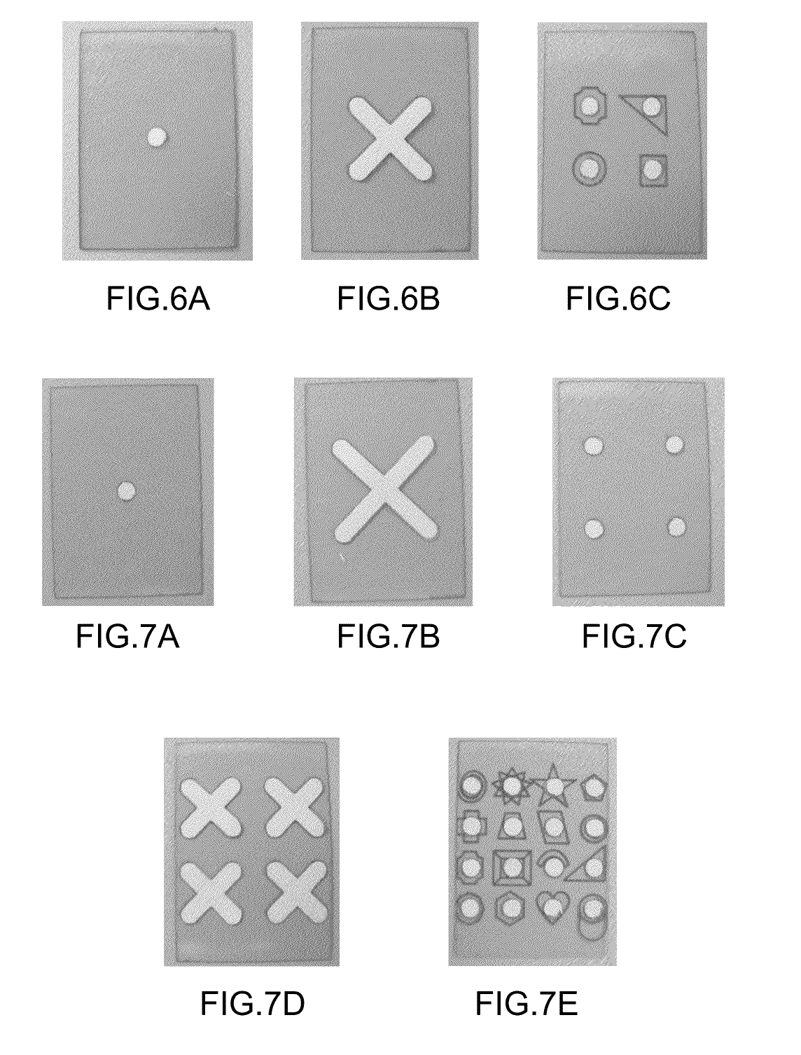

FIG. 6 presents the different stages (FIGS. 6A, 6B and 6C) with the 2^{nd} part nested into the 1^{st} support before assembly. In particular, FIGS. 6A, 6B and 6C respectively correspond to the dispensing, distribution and analysis stages of the solution of interest.

III.2. 16 Pad Reading Stage.

The different stages making up the 1^{st} support part consist of PET sheets 100 μm thick. The different stages have been obtained after cutting off with a CO₂ laser (Laser GCC Pro Spirit 25 watts). The design has been performed on Corel Draw 5 software.

The 2^{nd} part (fluidic system) has been obtained after cutting off CF1 Whatman paper by a CO₂ laser (Laser GCC Pro Spirit 25 watts). The design has been performed on Corel Draw 5 software.

FIG. 7 presents the different stages (FIGS. 7A, 7B, 7C, 7D and 7E) with the 2^{nd} part(s) nested into the 1^{st} support before assembly. In particular, FIGS. 7A, 7B, 7C, 7D and 7E respectively correspond to the dispensing, distribution, dispensing, distribution and analysis stages of the solution of interest.

III.3. Reading Stage for the 16 Pads pH.

The different stages making up the 1^{st} support part consist of PET sheets 100 μm thick. The different stages have been obtained after cutting off with a CO₂ laser (Laser GCC Pro Spirit 25 watts). The design has been performed on Corel Draw 5 software.

The 2^{nd} part (fluidic system) has been obtained after cutting off CF1 Whatman paper by a CO₂ laser (Laser GCC Pro Spirit 25 watts). The design has been performed on Corel Draw 5 software.

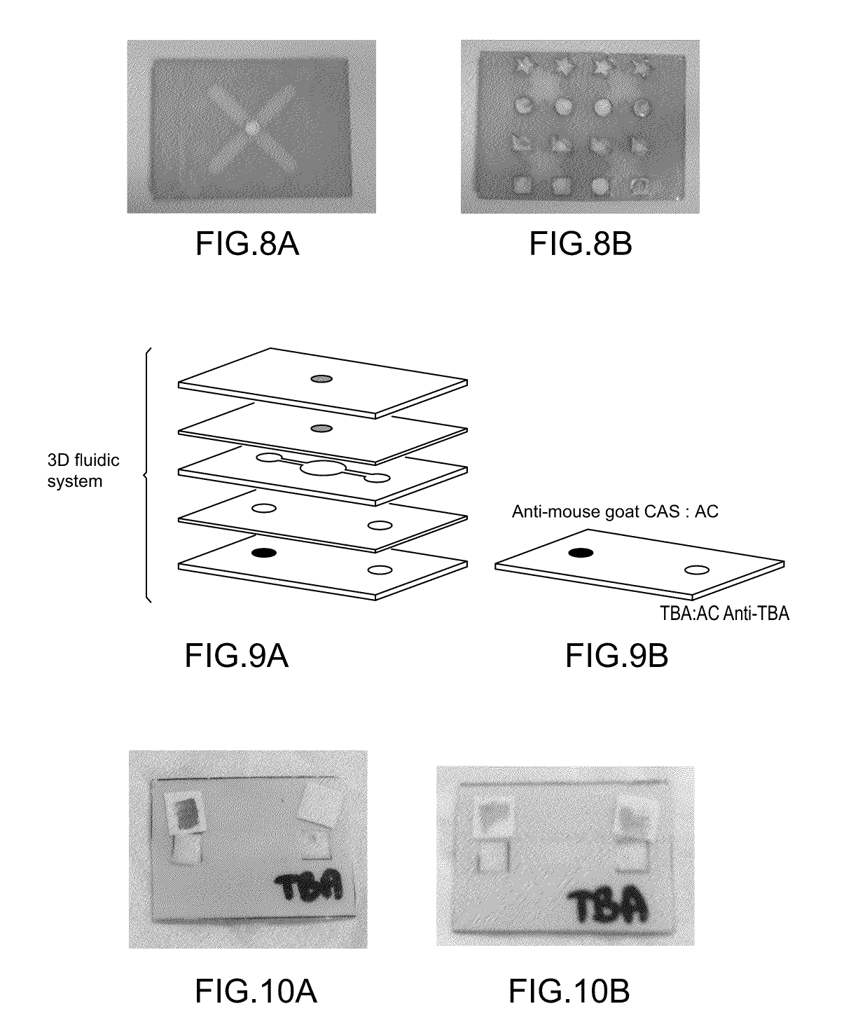

FIG. 8 presents a global system after assembly. FIG. 8A presents the top of the system at which the solution of interest to be analysed is introduced and FIG. 8B the stage for reading the results.

IV. Chemical Modification of Cellulose for Covalent Grafting of Biomolecules.

IV.1. Preliminary Remarks.

The 3D microfluidic systems according to the invention can be built-into the stage for reading the system.

To do this, the 2^{nd} part (i.e. the fluidic system which can be of cellulose for example), has to be modified in order to covalently graft therein biomolecules or organic molecules of interest which will enable information desired to be extracted from the solution of interest to be obtained.

Those skilled in the art know that it is extremely difficult to modify cellulose. These modifications generally occur on cellulose as fibrils under conditions incompatible with living organisms (organic solvent and high temperatures). Moreover, it is nearly impossible to work on shaped cellulose, for example as a sheet without destroying the macroscopic structure of the material.

In order to circumvent these problems, the inventors have developed methods allowing the modification under conditions compatible with living organisms of cellulose as sheets without altering the macroscopic structure. Two synthetic pathways have been used, the first based on a reduction into solutions of diazonium salts for which those skilled in the art can find further information in the International Application WO 2008/078052 [7] and the other based on a photochemical reaction at 385 nm using arylazide derivatives. Each of the pathways can occur preferentially in water or in biological buffer solutions but can also be used in solvent mixture systems or in an organic solvent. Both these pathways have allowed the introduction at the cellulose sheet of functions enabling biological molecules or organic molecules to be grafted under biologically compatible conditions, of the carboxylic acid, amine or thiol type.

In the same manner as described in the International Application WO 2008/078052 [7], a polymer having an interesting function within the scope of the present invention can be covalently grafted in cellulose without destructuring its macroscopic format and under conditions compatible with living organisms.

After modifying a cellulose sheet by these chemical methods, grafting of biomolecules is performed under conditions compatible with biology, well-known to those skilled in the art.

Of course, these methods can be also used for modifying intermediate stages of the system, if, for example, it is required to “actively” purify (i.e. a purification via in particular an antibody/antigen recognition and not by any “passive” type method such as the porosity size which allows a mechanical purification) or even to introduce any group necessary to the good working order of the 3D microfluidic system such as, for example, introducing anionic or cationic groups to obtain, from cellulose, a Sephadex resin equivalent.

IV.2. Grafting of Groups.

A. Grafting of Polyacrylic Acid on a Paper Sheet (Example A).

A GraftFast™ solution for grafting acrylic acid is created. For further information, the experimenter can refer to the International Application WO 2008/078052 [7].

A few drops of the solution thus obtained are deposited onto a 2 cm×2 cm piece of CF1 paper sheet from Whatman. After a time of 1 to 5 h, the paper is then rinsed on a frit by milliQ water (10 mL), and then by a 1 M soda solution (10 mL) and finally by milliQ water (10 mL) before being reacidified with a 1 M HCl solution (10 mL). The paper is then air dried. The ATR FTIR analysis does show the presence of the vibration band at 1710 cm⁻¹ corresponding to the vibration of the carboxylic acid group.

B. Grafting of a Poly(Carboxyl) Phenylene Layer on Paper (Example B).

4-aminobenzoic acid (Sigma Aldrich) (2 mmol, 274.3 mg) is transformed into diazonium salt by adding this compound into a 48% HBF₄ solution in the presence of 1.1 equivalent of NaNO₂ (2.2 mmol, 151.8 mg) with respect to the aniline derivative. The diazonium salt is obtained after precipitation in ether and filtration. The corresponding diazonium salt is dissolved in a 0.25 N HCl solution and then 2 mL of 50% weight H₃PO₂ are introduced in order to cause the reduction of the diazonium salts in solution (for further information, see [7]).

A few drops of the solution thus obtained are deposited onto a 2 cm×2 cm piece of CF3 paper sheet from Whatman. After a time of 1 to 6 h, the paper is then rinsed on a frit by milliQ water (10 mL), and then by a 1 M soda solution (10 mL) and finally by milliQ water (10 mL) before being reacidified with a 1 M HCl solution (10 mL). The paper is then air dried.

C. Grafting of a Poly(Amino) Phenylene Layer on Paper (Example C).

1,4-benzenediamine (Sigma Aldrich) (2 mmol, 216.3 mg) is transformed into diazonium salt by adding this compound into a 48% HBF₄ solution in the presence of 1.1 equivalent of NaNO₂ (2.2 mmol, 151.8 mg) with respect to the aniline derivative. The diazonium salt is obtained after precipitation in ether and filtration.

The corresponding diazonium salt is dissolved in a 0.25 N HCl solution and then 2 mL of 50% weight H₃PO₂ are introduced in order to cause the reduction of the diazonium salts in solution (for further information, see [7]).

A few drops of the solution thus obtained are deposited onto a 2 cm×2 cm piece of CF3 paper sheet from Whatman. After a time of 1 to 6 h, the paper is then rinsed on a frit by milliQ water (10 mL), and then by a 1 M soda solution (10 mL) and then by milliQ water (10 mL) and then reacidified with a 1 M HCl solution (10 mL). The paper is then air dried.

D. Grafting of a Poly(Ethyl Carboxyl)Phenylene Layer on Paper (Example D).

3-(4-aminophenyl)propionic acid (Sigma Aldrich) (2 mmol, 330.4 mg) is transformed into diazonium salt by adding this compound into a 48% HBF₄ solution in the presence of 1.1 equivalent of NaNO₂ (2.2 mmol, 151.8 mg) with respect to the aniline derivative. The diazonium salt is obtained after precipitation in ether and filtration.

The corresponding diazonium salt is dissolved in a 0.25 N HCl solution and then 2 mL of 50% weight H₃PO₂ are introduced in order to cause the reduction of the diazonium salts in solution (for further information, see [7]).

A few drops of the solution thus obtained are deposited onto a 2 cm×2 cm piece of CF3 paper sheet from Whatman. After a time of 1 to 6 h, the paper is then rinsed on a frit by milliQ water (10 mL), and then by a 1 M soda solution (10 mL) and then by milliQ water (10 mL) and then reacidified with a 1 M HCl solution (10 mL). The paper is then air dried.

E. Grafting of a Poly(Ethylamino) Phenylene Layer on Paper (Example E).

4-(2-aminoethyl)aniline (Sigma Aldrich) (2 mmol, 272.4 mg) is transformed into diazonium salt by adding this compound into a 48% HBF₄ solution in the presence of 1.1 equivalent of NaNO₂ (2.2 mmol, 151.8 mg) with respect to the aniline derivative.

The diazonium salt is obtained after precipitation in ether and filtration. The corresponding diazonium salt is dissolved in a 0.25 N HCl solution and then 2 mL of 50% weight H₃PO₂ are introduced in order to cause the reduction of the diazonium salts in solution (for further information, see [7]).

A few drops of the solution thus obtained are deposited onto a 2 cm×2 cm piece of CF3 paper sheet from Whatman. After a time of 1 to 6 h, the paper is then rinsed on a frit by milliQ water (10 mL), and then by a 1 M soda solution (10 mL) and then by milliQ water (10 mL) and then reacidified with a 1 M HCl solution (10 mL). The paper is then air dried.

F. Grafting of a Poly(Thiol) Phenylene Layer on Paper (Example F).

4-aminobenzenethiol (Sigma Aldrich) (2 mmol, 250.4 mg) is transformed into diazonium salt by adding this compound into a 48% HBF₄ solution in the presence of 1.1 equivalent of NaNO₂ (2.2 mmol, 151.8 mg) with respect to the aniline derivative. The diazonium salt is obtained after precipitation in ether and filtration.

The corresponding diazonium salt is dissolved in a 0.25 N HCl solution and then 2 mL of 50% weight H₃PO₂ are introduced in order to cause the reduction of the diazonium salts in solution (for further information, see [7]).

A few drops of the solution thus obtained are deposited onto a 2 cm×2 cm piece of CF3 paper sheet from Whatman. After a time of 1 to 6 h, the paper is then rinsed on a frit by milliQ water (10 mL), and then by a 1 M soda solution (10 mL) and then by milliQ water (10 mL) and then reacidified with a 1 M HCl solution (10 mL). The paper is then air dried.

G. Grafting of an Aminoaryl Layer on Paper (Example G).

1,4-benzenediamine (Sigma Aldrich) is transformed into diazonium salt by adding this compound into a 48% HBF₄ solution in the presence of 1.1 equivalent of NaNO₂ (2.2 mmol, 151.8 mg) with respect to the aniline derivative. The diazonium salt is obtained after precipitation in ether and filtration.

The corresponding diazonium salt is then resuspended in cold ether (4° C.) and then 4 equivalents of NaN₃ (4 mmol, 260 mg) are added and the solution comes back to the temperature for 3 h. The resulting solution is filtered in order to get rid of the salts. The ether is evaporated to dryness to give the corresponding arylazide.

The latter is dissolved in water or in a biological buffer such as PBS or TRIS sheltered from light. A few drops of the solution thus obtained are deposited onto a 2 cm×2 cm piece of CF3 or CF1 paper sheet from Whatman. The paper is then subjected to a 385 nm irradiation for a time ranging from 1 to 5 min. The paper is then rinsed on a frit by milliQ water (10 mL), and then by a 1 M soda solution (10 mL) and finally by milliQ water (10 mL) prior to being reacidified with a 1 M HCl solution (10 mL). The paper is then air dried.

H. Grafting of a Carboxylaryl Layer on Paper (Example H).

4-aminobenzoic acid (Sigma Aldrich) is transformed into diazonium salt by adding this compound into a 48% HBF₄ solution in the presence of 1.1 equivalent of NaNO₂ (2.2 mmol, 151.8 mg) with respect to the aniline derivative. The diazonium salt is obtained after precipitation in ether and filtration. The diazonium salt is then resuspended in cold ether (4° C.) and then 4 equivalents of NaN₃ (4 mmol, 260 mg) are added and the solution comes back to the temperature for 3 h. The resulting solution is filtered in order to get rid of the salts. The ether is evaporated dryness to give the corresponding arylazide.

The latter is dissolved in water or in a biological buffer such as PBS or TRIS sheltered from light. A few drops of the solution thus obtained are deposited onto a 2 cm×2 cm piece of CF3 or CF1 paper sheet from Whatman. The paper is then subjected to a 385 nm irradiation for a time ranging from 1 to 5 min. The paper is then rinsed on a frit by milliQ water (10 mL), and then by a 1 M soda solution (10 mL) and finally by milliQ water (10 mL) prior to being reacidified with a 1 M HCl solution (10 mL). The paper is then air dried.

J. Grafting of an Ethylaminoaryl Layer on Paper (Example J).

4-(2-aminoethyl)aniline (Sigma Aldrich) is transformed into diazonium salt by adding this compound into a 48% HBF₄ solution in the presence of 1.1 equivalent of NaNO₂ (2.2 mmol, 151.8 mg) with respect to the aniline derivative. The diazonium salt is obtained after precipitation in ether and filtration.

The diazonium salt is then resuspended in cold ether (4° C.) and then 4 equivalents of NaN₃ (4 mmol, 260 mg) are added and the solution comes back to the temperature for 3 h. The resulting solution is filtered in order to get rid of the salts. The ether is evaporated to dryness to give the corresponding arylazide.

The latter is dissolved in water or in a biological buffer such as PBS or TRIS sheltered from light. A few drops of the solution thus obtained are deposited onto a 2 cm×2 cm piece of CF3 or CF1 paper sheet from Whatman. The paper is then subjected to a 385 nm irradiation for a time ranging from 1 to 5 min. The paper is then rinsed on a frit by milliQ water (10 mL), and then by a 1 M soda solution (10 mL) and finally by milliQ water (10 mL) prior to being reacidified with a 1 M HCl solution (10 mL). The paper is then air dried.

K. Grafting of an Ethylcarboxylaryl Layer on Paper (Example K).

3-(4-aminophenyl)propionic acid (Sigma Aldrich) is transformed into diazonium salt by adding this compound into a 48% HBF₄ solution in the presence of 1.1 equivalent of NaNO₂ (2.2 mmol, 151.8 mg) with respect to the aniline derivative. The diazonium salt is obtained after precipitation in ether and filtration.

The diazonium salt is then resuspended in cold ether (4° C.) and then 4 equivalents of NaN₃ (4 mmol, 260 mg) are added and the solution comes back to the temperature for 3 h. The resulting solution is filtered in order to get rid of the salts. The ether is evaporated to dryness to give the corresponding arylazide.

The corresponding arylazide is dissolved in water or in a biological buffer such as PBS or TRIS sheltered from light. A few drops of the solution thus obtained are deposited onto a 2 cm×2 cm piece of CF3 or CF1 paper sheet from Whatman. The paper is then subjected to a 385 nm irradiation for a time ranging from 1 to 5 min. The paper is then rinsed on a frit by milliQ water (10 mL), and then by a 1 M soda solution (10 mL) and finally by milliQ water (10 mL) prior to being reacidified with a 1 M HCl solution (10 mL). The paper is then air dried.

L. Grafting of a Thioaryl Layer on Paper (Example L).

4-aminobenzenethiol (Sigma Aldrich) is transformed into diazonium salt by adding this compound into a 48% HBF₄ solution in the presence of 1.1 equivalent of NaNO₂ (2.2 mmol, 151.8 mg) with respect to the aniline derivative. The diazonium salt is obtained after precipitation in ether and filtration.

The diazonium salt is then resuspended in cold ether (4° C.) and then 4 equivalents of NaN₃ (4 mmol, 260 mg) are added and the solution comes back to the temperature for 3 h. The resulting solution is filtered in order to get rid of the salts. The ether is evaporated to dryness to give the corresponding arylazide.

The corresponding arylazide is dissolved in water or in a biological buffer such as PBS or TRIS sheltered from light. A few drops of the solution thus obtained are deposited onto a 2 cm×2 cm piece of CF3 or CF1 paper sheet from Whatman. The paper is then subjected to a 385 nm irradiation for a time ranging from 1 to 5 min. The paper is then rinsed on a frit by milliQ water (10 mL), and then by a 1 M soda solution (10 mL) and finally by milliQ water (10 mL) prior to being reacidified with a 1 M HCl solution (10 mL). The paper is then air dried.

IV.3. Grafting of Antibodies.

a. On the Papers of Examples A, B, C, D, E, F, G, H and J.

Anti-ricin, anti-enterotoxin B of Staphylococcus Aureus, anti-Bacillus antracis, anti-ovalbumin and anti-beta-lactoglobulin antibodies in solution in PBS buffer (1 mg/mL) are contacted (200 μL) with the papers of Examples A, B, C, D, E, F, G, H and J in the presence of an EDC/NHS (for “1-ethyl-3-(3-dimethylaminopropyl)carbodiimide/N-hydroxysuccinimide) 50 mM solution and are incubated with the papers for 12 h at 4° C. The papers are then rinsed with PBS buffer and then they are blocked with a BSA solution in PBS.

B. On the Papers of Examples F and L.

Anti-ricin, anti-enterotoxin B of Staphylococcus Aureus, anti-Bacillus antracis, anti-ovalbumin and anti-beta-lactoglobulin antibodies in solution in PBS buffer (1 mg/mL) are contacted (200 μL) with the papers of Examples F and L and are incubated for 12 h at 4° C. The papers are then rinsed with PBS buffer and then they are blocked with a BSA solution in PBS.

IV.4. Grafting on Paper of Antibodies Modified by an Arylazide Arm.

Anti-ricin, anti-enterotoxin B of Staphylococcus Aureus, anti-Bacillus antracis, anti-ovalbumin and anti-beta-lactoglobulin antibodies are modified with linkers possessing an arylazide group as the ANB-NOS compound or the Sulfo-SAN PAH compound (Pierce, Thermo Fisher) of the formula:

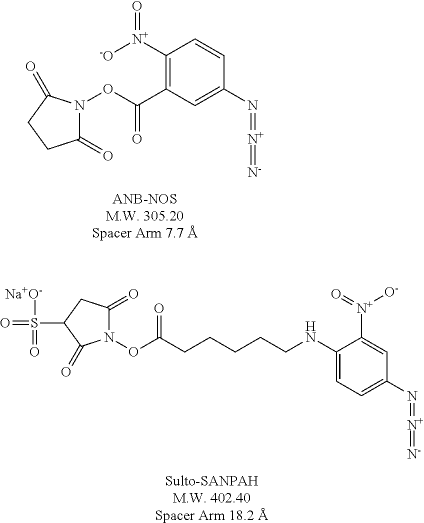

Thus obtained antibodies are purified by chromatography. CF1 Whatman paper is soaked with 100 μL of a solution of these modified antibodies (1 mg/mL) with the arylazide group and then the samples are subjected to a 385 nm irradiation for a time ranging from 1 to 5 min.

It should be noted that the use of these photoactivable groups enables the biomolecules and their biological activity not to be modified nor altered. After the photochemical reaction, the papers are rinsed with PBS buffer (1 mL).

V. Use of a 3D Microfluidic System According to the Invention for Detecting Botulinum Toxin A.

V.1. Design of the System.

FIG. 9A presents the different layers of the 3D microfluidic system according to the invention.

By referring to the layers such as described for the system of point I, the system implemented for detecting the botulinum toxin comprises layers (1) to (5) with the support part of layers (1), (3) and (5) of PET polymer and the fluidic channel type part(s) of the layers (3) and (5) of cellulose or nitrocellulose.

The layers (2) and (4) are made of double sided Scotch® tape with the recess of layer (2) left as such, whereas, in the recesses of the layer (4), parts of cellulose or nitrocellulose have been nested.

As previously described, the polymer sheets and cellulose sheets are cut off according to the same pattern, the one being the negative of the other. All the sheets are held using a double sided Scotch® tape.

Finally, as presented in FIG. 9B, the layer (5) corresponding to the analysis stage presents functionalized fluidic channel type parts, for one, by an anti-botulinum toxin A antibody produced by CEA and, for the other one, hereinafter designated “control pad”, by an anti-mouse goat antibody (Jackson Immunoresearch Laboratories Inc; Code 115-005-004). The fluidic channel part of the analysis stage (i.e. layer (5)) is of nitrocellulose and the different antibodies have been directly immobilized on the same, by electrostatic interaction without any step other than the deposition of antibody solution.

V.2. Results Obtained.

Two different samples have been tested on microfluidic systems such as prepared at point III.1.

The 1^{st} sample contains an anti-botulinum toxin A mouse antibody labelled with colloidal gold (1 μg; antibody and labelling performed at CEA, Volland et al., 2007 [9])) without a botulinum toxin A in a 0.1 M potassium phosphate buffer (pH 7.4)+0.15 M NaCl+1 mg/ml bovine serum albumin+0.01% sodium azide. FIG. 10A presents the result obtained following the deposition of a 500 μl drop of the 1^{st} sample, only the control pad being coloured.

The 2^{nd} sample contains an anti-botulinum toxin A mouse antibody labelled with colloidal gold (1 μg; antibody and labelling performed at CEA, Volland et al., 2007 [9])) and a non-toxic region of botulinum toxin A recognized by the antibody (100 ng/ml, recombinant protein corresponding to the binding domain of botulinum toxin A; the preparation of this recombinant protein being described by Tavallaie et al., 2004 [10]) in a 0.1 M potassium phosphate buffer (pH 7.4)+0.15 M NaCl+1 mg/ml bovine serum albumin+0.01% sodium azide. FIG. 10B presents the result obtained following the deposition of a 500 μl drop of the 2^{st} sample, both pads being coloured.

These results show that the detection of a toxin at the reaction zone can be done.

VI. Preparation of a 3D Microfluidic System Having a Built-in Reservoir and a Double Spiral According to the Invention.

The 3D microfluidic system having a built-in reservoir according to the present invention described hereinafter is a system adapted for the hydrophilic solutions of interest.

The materials implemented and the manufacture of the support parts and the “fluidic channel” type parts are such as defined in paragraphs 1.1 and 1.2 above.

VI.1. Manufacture of the Built-in Reservoir.

A hollow pattern is made on different stages of the device. This hollow pattern is not subsequently filled with a hydrophilic porous material, the stack of stages thus enables a cavity which is contacting with a hydrophilic porous material to be created. The sample is deposited into the reservoir and then, by capillarity, migrates into the hydrophilic porous material.

VI.2. Manufacture of the Spiral Pattern.

The spiral or double spiral fluidic circuit is manually cut off or using a CO₂ laser printer:
in the hydrophobic or non-porous matrix, the matrix is recovered with its spiral or double spiral cut off which makes up the “negative of the device”;
in the porous hydrophilic material, the spiral or double spiral cut off part is recovered, which makes up the “positive” of the device and
in the adhesive tape, the adhesive is recovered with its cut off.

VI.3. Assembly of the System and Thus Obtained System.

The assembly of the device is made by

1/ nesting the cut off hydrophilic part (“positive”) in the hydrophobic material (“negative”), the assembly making up a mixed layer;

2/ bonding onto the mixed layer surface of the cut off double sided adhesive tape;

3/ positioning a new mixed layer, a mixed layer comprising at least one unfilled recessed zone or a support layer of hydrophobic material none the recesses of which is filled on the adhesive surface,

steps 2 and 3 can be repeated as many times as required.

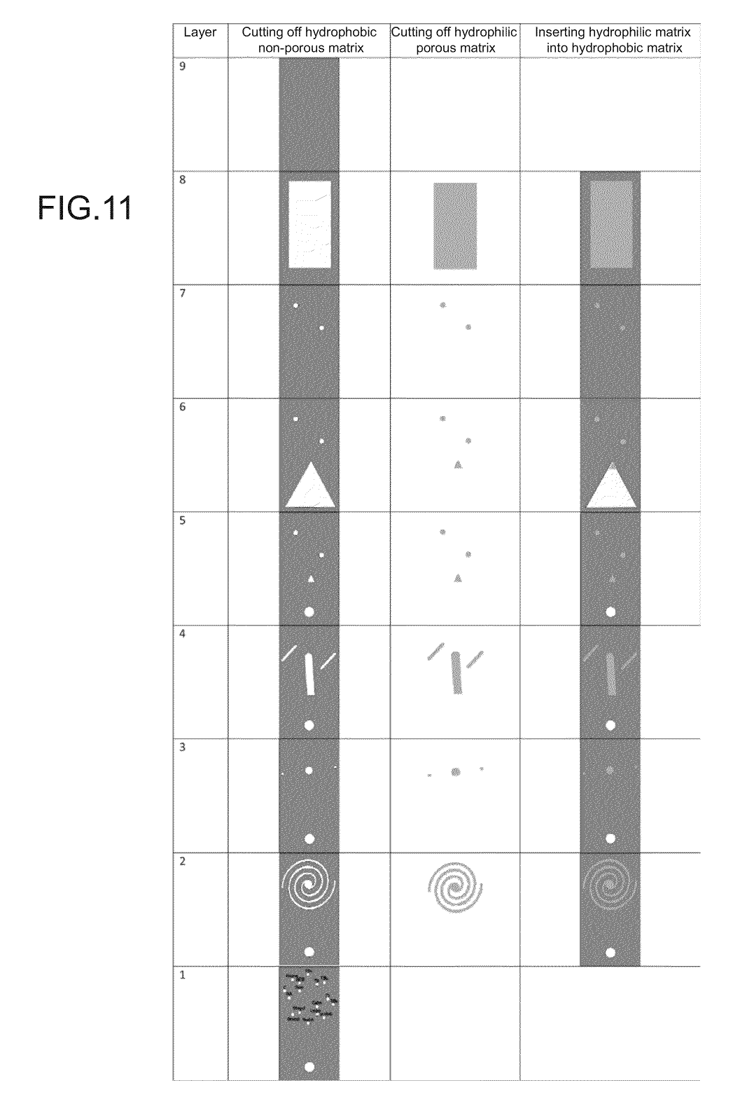

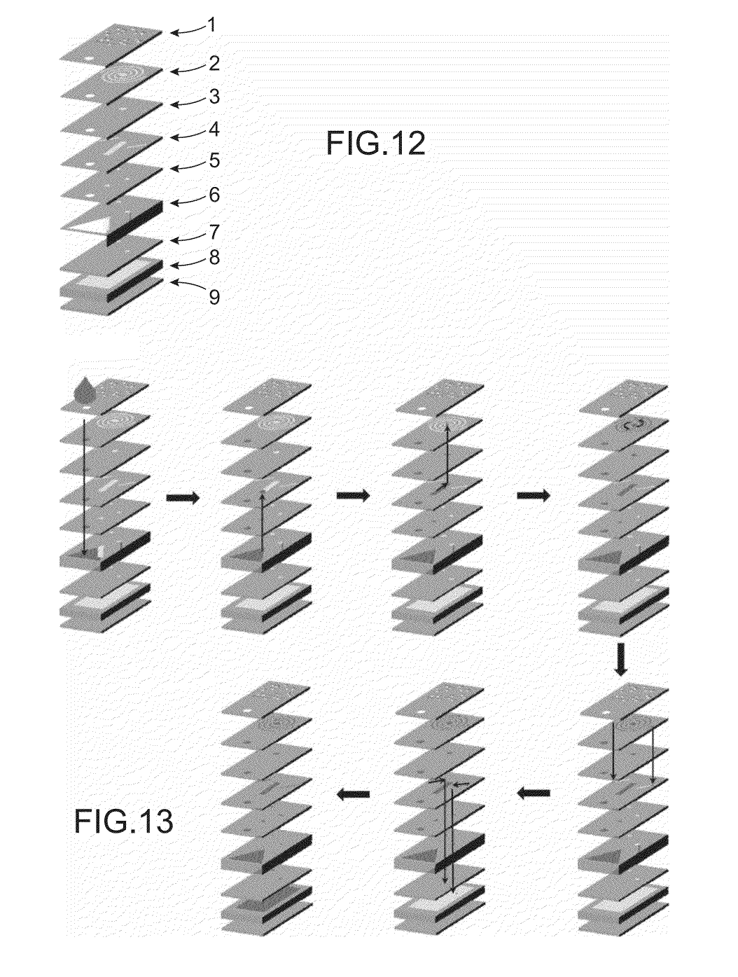

The different elements making up the layers of the 3D microfluidic system having a built-in reservoir and a spiral fluidic circuit and the assembled layers obtained are presented in FIG. 11 and the final microfluidic system is presented in FIG. 12. It should be noted that the layers corresponding to the double sided Scotch® tape possibly present between two layers illustrated in FIGS. 11 and 12 are not represented on the same.

Layers (1) and (9) of this system do not make up layers with a 1^{st} and at least one 2^{nd} parts such as defined in the present invention. Indeed, none of the recesses of the layer (1) is filled by a hydrophilic porous part. This layer (1) is, on the one hand, the layer at which the sample of the solution of interest to be analysed or purified represented by a drop is introduced via an unfilled circular recess and, on the other hand, the layer of which other unfilled recesses facing detection zones made on the double spiral of the layer (2) allow the visualisation of the signal which reveals or not the presence of the agent searched for. Moreover, the support layer (9) is left as such contributing, de facto, to support the hydrophilic parts of the upper stages.

Furthermore, the system of FIGS. 11 and 12 is characterized by:

(i) at the layer (2), an analysis double spiral of porous hydrophilic material at which detection zones have been made in particular by implementing one or more of the protocols described at paragraph IV;

(i) at the layer (6), a reservoir the base of which is formed by a part of the support of the layer (7) and the walls by the edges of one of the recesses of the layer (6), the reservoir recess, although unfilled, having an element of a hydrophilic porous material, and

(iii) at the layer (8), a hydrophilic matrix corresponds to absorb paper which keeps the movement of the sample in the device by capillarity and which preserves it once the latter has flowed on the entire device, the hydrophilic matrix of this layer (8) can be considered, therefore, as a “biological dustbin” or as a low-cost system playing the role of a “pump”.

The layers (1), (2), (3), (4) and (5) all have an unfilled recess, the unfilled recesses of these layers being centred on a common axis, and having identical diameter whereby they form a cannula type channel enabling the sample of solution of interest to be analysed or purified to be led from the layer (1) to the reservoir of the layer (6).

Further, the layers (3), (4) and (5) all have a 1^{st} recess filled by a hydrophilic porous material enabling the conveyance by capillarity of the sample of solution of interest to be analysed or purified from the reservoir of the layer (6) to the double spiral of the layer (2). The hydrophilic porous material of this 1^{st} filled recess at the layer (5) extends from the hydrophilic porous material present at the reservoir of the layer (6). These 1^{st} recesses do not have identical shapes from one layer to another and are not necessarily centred on a common axis, the only condition being that there is a fluidic continuity between the hydrophilic porous materials filling them.

Finally, the layers (3), (4), (5), (6) and (7) each have two recesses having an identical shape and filled by a hydrophilic porous material, advantageously with a same hydrophilic porous material; these two recesses each in the fluidic continuity of one of the ends of the double spiral of the layer (2) enabling the sample of solution of interest to conveyed by capillarity, after contacting the spiral of hydrophilic porous material of the layer (2) to the hydrophilic material of the layer (8). These recesses do not have identical shapes from one layer to another and are not necessarily centred on two common axes, the only condition being that there is a 1^{st} fluidic continuity between the hydrophilic porous materials which fill the 1^{st} of these recesses and a 2^{nd} fluidic continuity between the hydrophilic porous materials which fill the 2^{nd} of these recesses.

Thus, in the system presented in FIG. 12, the sample of interest is contacted with or passes through several layers several times, a 1^{st} time to go to the reservoir of the layer (6) (descending direction), a 2^{nd} time to go from the reservoir of the layer (6) to the analysis layer (2) (ascending direction) and a 3^{rd} and last time to go from the analysis layer (2) to the “dustbin” layer (8). FIG. 13 chronologically presents the movement of the sample in the system of FIG. 12.

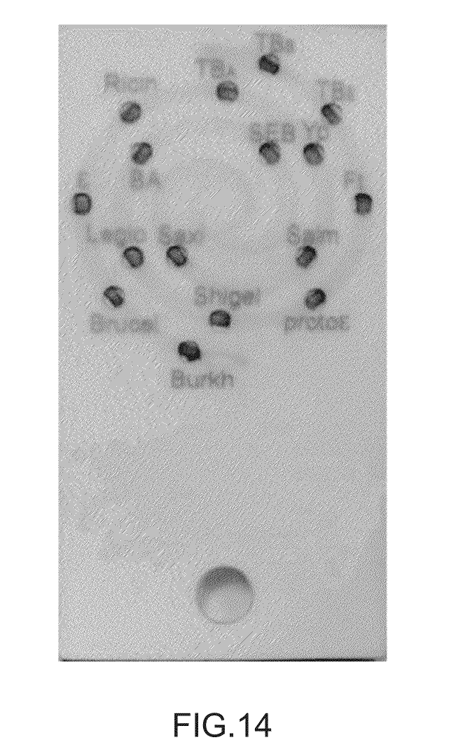

As previously explained, the double spiral of the layer can be functionalized by different reagents or compounds able to detect or trap a component or analyte possibly present in the solution of interest to be analysed. More particularly, this functionalization by different reagents or compounds is made at particular zones of the double spiral. FIG. 14 presents information which can be obtained by looking at the layer (1) of the system of FIG. 12. Thus, from a sample of interest deposited onto the system according to the invention which is divided into two only at the double spiral, it is possible to obtain information for 16 components or analytes present or not in this sample (16 pads of FIG. 14).

### BIBLIOGRAPHY

1. International Application WO 2009/121037, on behalf of President and Fellows of Harvard College, published on Oct. 1, 2009.
2. Martinez, A. et al., 2008, “Three-dimensional microfluidic devices fabricated in layered paper and tape”, PNAS, vol. 105, pages 19606-19611.
3. Li, X. et al., “Fabrication of paper-based microfluidic sensors by printing”, Colloids and Surfaces B: Biointerfaces, pages 564-570.
4. Lu, Y. et al., 2010, “Fabrication and characterization of paper-based microfluidics prepared in nitrocellulose membrane by wax printing”, Anal. Chem., vol. 82, pages 329-335.
5. International Application WO 2011/000047, on behalf of Monash University, published on Jan. 6, 2011.
6. International Application WO 2010/003188, on behalf of Monash University, published on Jan. 14, 2010.
7. International Application WO 2008/078052, on behalf of CEA, published on Jul. 3, 2008.
8. International Application WO 2009/1211944, on behalf of CEA, published on Oct. 8, 2009.
9. Volland H., et al., 2008, “A sensitive sandwich enzyme immunoassay for free or complexed Clostridium botulinum neurotoxin type A.”, J. Immunol. Methods, vol. 330, pages 120-129.
10. Tavallaie, M., et al., 2004. “Interaction between the two subdomains of the C-terminal part of the botulinum neurotoxin A is essential for the generation of protective antibodies.”, FEBS Lett., vol. 572, page 299.

1-19. (canceled)

20. A three dimensional (3D) microfluidic system comprising a plurality of layers stacked upon each other, wherein at least one of said layers consists of a 1^{st} and at least one 2^{nd} part, distinct from each other, with the previously cut off 2^{nd} part being porous and wettable by a solution of interest, nesting into a recess of the 1^{st} part being non-porous and/or non-wettable by said solution of interest, said 2^{nd} part acting as a fluidic channel providing transfer of said solution of interest by a capillarity effect into the plane and/or thickness of said system.

21. The 3D microfluidic system according to claim 20, wherein said 1^{st} part is:
porous and hydrophobic;
non-porous and hydrophilic; or
non-porous and hydrophobic; and
said 2^{nd} part(s) is hydrophilic and porous.

22. The 3D microfluidic system according to claim 21 wherein said 1^{st} part is selected from the group consisting of a porous or non-porous film of polyethylene terephthalate (PET); a porous or non-porous membrane of polyethylene (PE); a porous or non-porous membrane of polypropylene (PP); a porous or non-porous film or a porous or non-porous membrane containing fluorine; a porous or non-porous membrane of polytetrafluoroethylene (PTFE); a porous or non-porous copolymeric film comprising vinylidene fluoride and tetrafluoroethylene; a porous or non-porous copolymeric film comprising vinylidene fluoride and hexafluoropropylene; a porous or non-porous film of polymethyl methacrylate (PMMA); a porous or non-porous film of poly(n-butyl acetate); a porous or non-porous film of poly(benzyl methacrylate); a porous or non-porous film of poly(chlorotrifluoroethylene); an ion exchange porous membrane, functionalized by hydrophobic groups; a styrene polymer; a porous or non-porous film of polyacrylonitrile; a porous or non-porous film of polymethacrylonitrile; a porous or non-porous film of polyimide; and a mixture thereof.

23. The 3D microfluidic system according to claim 21, wherein said 2^{nd} part is selected from the group consisting of paper of cellulosic type; cotton paper; agarose; gelatin; cellulose; methylcellulose; carboxymethylcellulose; nitrocellulose; cellulose acetate ester; alginate; polyolefin; an ion exchange porous membrane functionalized by hydrophilic groups; a Sephadex type resin conditioned as a membrane or a PVDF membrane; a glass fibre tissue; a polyacrylamide gel; a sepharose gel; and a mixture thereof.

24. The 3D microfluidic system according to claim 20, wherein said 1^{st} part is:
porous and hydrophilic;
non-porous and hydrophilic; or
non-porous and hydrophobic; and
said 2^{nd} part(s) is hydrophobic and porous.

25. The 3D microfluidic system according to claim 24, wherein said 1^{st} part is selected from the group consisting of paper; cotton paper; agarose; gelatin; cellulose; methylcellulose; carboxymethylcellulose; nitrocellulose; cellulose acetate ester; alginate; polyolefin; an ion exchange membrane advantageously functionalized by radiochemical grafting, by hydrophilic groups; a Sephadex type resin conditioned as a membrane or a PVDF membrane; a glass fibre tissue; a non-porous film of polyethylene terephthalate (PET); a non-porous membrane of polyethylene (PE); a non-porous membrane of polypropylene (PP); a non-porous film of polyimide; a polyacrylamide gel; a sepharose gel; and a mixture thereof.

26. The 3D microfluidic system according to claim 24, wherein said 2^{nd} part is selected from the group consisting of paper hydrophobized by treatment, a porous film of polyethylene terephthalate (PET); a porous membrane of polyethylene (PE); a porous membrane of polypropylene (PP); a porous film or a porous membrane containing fluorine; a porous membrane of polytetrafluoroethylene (PTFE); a porous copolymeric film containing vinylidene fluoride and tetrafluoroethylene; a porous copolymeric film comprising vinylidene fluoride and hexafluoropropylene; a polyacrylamide gel; a porous film of polymethyl methacrylate (PMMA); a porous film of poly(n-butyl acetate); a porous film of poly(benzyl methacrylate); a porous film of poly(chlorotrifluoroethylene); an ion exchange porous membrane, functionalized by hydrophobic groups; a porous styrene polymer; a porous film of polyacrylonitrile; a porous film of polymethacrylonitrile; and a mixture thereof.

27. The 3D microfluidic system according to claim 20, wherein at least one layer consisting of a 1^{st} part and at least one 2^{nd} part, distinct from each other, with the 2^{nd} part being porous and wettable by a solution of interest nesting into a recess of the 1^{st} part being non-porous and/or non-wettable by said solution of interest, said 1^{st} part further has at least one unfilled recess.

28. The 3D microfluidic system according to claim 20, wherein the system comprises at least one layer consisting only of a support being non-porous and/or non-wettable by a solution of interest and recessed at one or more defined zones.

29. The 3D microfluidic system according to claim 20, wherein the system comprises at least one layer of the at least a 2^{nd} part of which plays the role of a biological dustbin.

30. The 3D microfluidic system according to claim 20, wherein the system comprises at least one layer of the at least a 2^{nd} part of which has a spiral or double spiral shape.

31. The 3D microfluidic system according to claim 20, wherein the system comprises, between at least two consecutive layers, an element able to secure these two layers together and/or ensure tightness between these two layers.

32. The 3D microfluidic system according to claim 31, wherein said element comprises a double sided Scotch® tape, a Saran microwave stretchable film type stretchable film or a derivative of a supported adhesion primary coat.

33. A device comprising a 3D microfluidic system as defined in claim 20.

34. A method for manufacturing a 3D microfluidic system as defined in claim 20, said method comprising the steps of:
(a₁) preparing, in a layer of a material being non-porous and/or non-wettable by a solution of interest, a recess;
(b₁) cutting off, in a layer of a material being porous and wettable by said solution of interest, at least one shape in accordance with the recess prepared in step (a₁);
(c₁) nesting into the recess prepared in step (a₁) the shape cut off in step (b₁), whereby a layer of said 3D microfluidic system is obtained;
(d₁) optionally repeating steps (a₁), (b₁) and (c₁); and
(e₁) assembling the different layers of the 3D microfluidic system.

35. The manufacturing method according to claim 34, wherein, during said step (a₁), said recess has a cross-section with respect to the plane of the layer in a spiral or double spiral shape.

36. A method for detecting and optionally quantifying at least one analyte possibly present in a solution of interest or in a gas fluid of interest, said method comprising the steps of:
depositing a solution of interest onto either a 3D microfluidic system comprising a plurality of layers stacked upon each other, wherein at least one of said layers consists of a 1^{st} and at least one 2^{nd} part, distinct from each other, with the previously cut off 2^{nd} part being porous and wettable by a solution of interest, nesting into a recess of the 1^{st} part being non-porous and/or non-wettable by said solution of interest, said 2^{nd} part acting as a fluidic channel providing transfer of said solution of interest by a capillarity effect into the plane and/or thickness of said system, or
a 3D microfluidic system prepared by a manufacturing method according to claim 34 or
contacting a gas fluid of interest with either the 3D microfluidic system comprising a plurality of layers stacked upon each other or a 3D microfluidic system prepared by a manufacturing method according to claim 34, and
detecting and optionally quantifying said analyte possibly present.

37. A method for purifying at least one analyte possibly present in a solution of interest or in a gas fluid of interest, said method comprising the steps of:
depositing a solution of interest onto either a 3D microfluidic system comprising a plurality of layers stacked upon each other, wherein at least one of said layers consists of a 1st and at least one 2^{nd} part, distinct from each other, with the previously cut off 2^{nd} part being porous and wettable by a solution of interest, nesting into a recess of the 1^{st} part being non-porous and/or non-wettable by said solution of interest, said 2^{nd} part acting as a fluidic channel providing transfer of said solution of interest by a capillarity effect into the plane and/or thickness of said system or
a 3D microfluidic system prepared by a manufacturing method according to claim 34 or
contacting a gas fluid of interest with the 3D microfluidic system comprising a plurality of layers stacked upon each other or with a 3D microfluidic system prepared by a manufacturing method according to claim 34, and
purifying said analyte possibly present.

38. A method according to claim 36 wherein said analyte is selected from the group consisting of a biological molecule of interest; a pharmacological molecule of interest; a toxin; a carbohydrate; a peptide; a protein; a glycoprotein; an enzyme; an enzymatic substrate; a nuclear or membrane receptor; an agonist or antagonist of a nuclear or membrane receptor; a hormone; a polyclonal or monoclonal antibody; an antibody fragment comprising a Fab, F(ab′)₂, Fv fragment or a hypervariable domain or CDR (Complementarity Determining Region); a nucleotide molecule; an advantageously organic pollutant of water; an advantageously organic pollutant of air; a bacterium and a virus.

39. A method according to claim 37, wherein said analyte is selected from the group consisting of a biological molecule of interest; a pharmacological molecule of interest; a toxin; a carbohydrate; a peptide; a protein; a glycoprotein; an enzyme; an enzymatic substrate; a nuclear or membrane receptor; an agonist or antagonist of a nuclear or membrane receptor; a hormone; a polyclonal or monoclonal antibody; an antibody fragment comprising a Fab, F(ab′)₂, Fv fragment or a hypervariable domain or CDR (Complementarity Determining Region); a nucleotide molecule; an advantageously organic pollutant of water; an advantageously organic pollutant of air; a bacterium and a virus.
